SEQUENCE LISTING (23 sequences)

SEQ ID NO: 1 - DNA, 753 bp, Homo sapiens
  CDS (1)..(753)
atggaggtgcagcttcaggagtcaggacctagcctcgtgaaaccttctcagactctgtccctcacctgttctgtcactggcgactccatcaccagtggttactggaactggatccggaagttcccagggaataaacttgaatatatgggttacataagcgacagtggtatcacttactacaatccatctctcaaaagtcgcatttccatcactcgagacacatccaagaaccaatactacctccagttgaattttgtgactgctgaggacacagccacatataactgtgcaagaaggactctggctacttactatgctatggactactggggtcaaggaacctctgtcaccgtctcctcaggctcgacgtccggctctggcaaaccgggctctggcgaaggctctaccaagggcgattttgtgatgacccaaactccactctccctgcctgtcagtcttggagatcaagcctccatctcttgcagggctagtcagagccttgtacacagtaatggaaacacctatttacattggtacctgcagaagccaggccagtctccaaagctcctgatctacagagtttccaaccgattttctggggtcccagacaggttcagtggcagtggatcagggacagatttcacactcaagatcagcagagtggaggctgaggatctgggagtttatttctgctctcaaagtacacatgttcctccgacgttcggtggaggcaccaagctggaaatcaaacgg

SEQ ID NO: 2 - protein, 251 aa, Homo sapiens
MEVQLQESGPSLVKPSQTLSLTCSVTGDSITSGYWNWIRKFPGNKLEYMGYISDSGITYYNPSLKSRISITRDTSKNQYYLQLNFVTAEDTATYNCARRTLATYYAMDYWGQGTSVTVSSGSTSGSGKPGSGEGSTKGDFVMTQTPLSLPVSLGDQASISCRASQSLVHSNGNTYLHWYLQKPGQSPKLLIYRVSNRFSGVPDRFSGSGSGTDFTLKISRVEAEDLGVYFCSQSTHVPPTFGGGTKLEIKR

SEQ ID NO: 3 - DNA, 1839 bp, Homo sapiens
  CDS (1)..(1839)
atggaggtgcagcttcaggagtcaggacctagcctcgtgaaaccttctcagactctgtccctcacctgttctgtcactggcgactccatcaccagtggttactggaactggatccggaagttcccagggaataaacttgaatatatgggttacataagcgacagtggtatcacttactacaatccatctctcaaaagtcgcatttccatcactcgagacacatccaagaaccaatactacctccagttgaattttgtgactgctgaggacacagccacatataactgtgcaagaaggactctggctacttactatgctatggactactggggtcaaggaacctctgtcaccgtctcctcaggctcgacgtccggctctggcaaaccgggctctggcgaaggctctaccaagggcgattttgtgatgacccaaactccactctccctgcctgtcagtcttggagatcaagcctccatctcttgcagggctagtcagagccttgtacacagtaatggaaacacctatttacattggtacctgcagaagccaggccagtctccaaagctcctgatctacagagtttccaaccgattttctggggtcccagacaggttcagtggcagtggatcagggacagatttcacactcaagatcagcagagtggaggctgaggatctgggagtttatttctgctctcaaagtacacatgttcctccgacgttcggtggaggcaccaagctggaaatcaaacggacgccagtgtcagaaaaacagctggcggaggtggtcgcgaatacgattaccccgctgatgaaagcccagtctgttccaggcatggcggtggccgttatttatcagggaaaaccgcactattacacatttggcaaggccgatatcgcggcgaataaacccgttacgcctcagaccctgttcgagctgggttctataagtaaaaccttcaccggcgttttaggtggggatgccattgctcgcggtgaaatttcgctggacgatgcggtgaccagatactggccacagctgacgggcaagcagtggcagggtattcgtatgctggatctcgccacctacaccgctggcggcctgccgctacaggtaccggatgaggtcacggataacgcctccctgctgcgcttttatcaaaactggcagccgcagtggaagcctggcacaacgcgtctttacgccaacgccagcatcggtctttttggtgcgctggcggtcaaaccttctggcatgccctatgagcaggccatgacgacgcgggtccttaagccgctcaagctggaccatacctggattaacgtgccgaaagcggaagaggcgcattacgcctggggctatcgtgacggtaaagcggtgcgcgtttcgccgggtatgctggatgcacaagcctatggcgtgaaaaccaacgtgcaggatatggcgaactgggtcatggcaaacatggcgccggagaacgttgctgatgcctcacttaagcagggcatcgcgctggcgcagtcgcgctactggcgtatcgggtcaatgtatcagggtctgggctgggagatgctcaactggcccgtggaggccaacacggtggtcgagacgagttttggtaatgtagcactggcgccgttgcccgtggcagaagtgaatccaccggctcccccggtcaaagcgtcctgggtccataaaacgggctctactggcgggtttggcagctacgtggcctttattcctgaaaagcagatcggtattgtgatgctcgcgaatacaagctatccgaacccggcacgcgttgaggcggcataccatatcctcgaggcgctacagtag

SEQ ID NO: 4 - protein, 612 aa, Homo sapiens
MEVQLQESGPSLVKPSQTLSLTCSVTGDSITSGYWNWIRKFPGNKLEYMGYISDSGITYYNPSLKSRISITRDTSKNQYYLQLNFVTAEDTATYNCARRTLATYYAMDYWGQGTSVTVSSGSTSGSGKPGSGEGSTKGDFVMTQTPLSLPVSLGDQASISCRASQSLVHSNGNTYLHWYLQKPGQSPKLLIYRVSNRFSGVPDRFSGSGSGTDFTLKISRVEAEDLGVYFCSQSTHVPPTFGGGTKLEIKRTPVSEKQLAEVVANTITPLMKAQSVPGMAVAVIYQGKPHYYTFGKADIAANKPVTPQTLFELGSISKTFTGVLGGDAIARGEISLDDAVTRYWPQLTGKQWQGIRMLDLATYTAGGLPLQVPDEVTDNASLLRFYQNWQPQWKPGTTRLYANASIGLFGALAVKPSGMPYEQAMTTRVLKPLKLDHTWINVPKAEEAHYAWGYRDGKAVRVSPGMLDAQAYGVKTNVQDMANWVMANMAPENVADASLKQGIALAQSRYWRIGSMYQGLGWEMLNWPVEANTVVETSFGNVALAPLPVAEVNPPAPPVKASWVHKTGSTGGFGSYVAFIPEKQIGIVMLANTSYPNPARVEAAYHILEALQ

SEQ ID NO: 5 - DNA, 27 bp, Artificial
  Description of Artificial Sequence Primer
accaagggcgatgttgtgatgacccaa

SEQ ID NO: 6 - DNA, 27 bp, Artificial
  Description of Artificial Sequence Primer
ttgggtcatcacaacatcgcccttggt

SEQ ID NO: 7 - DNA, 27 bp, Artificial
  Description of Artificial Sequence Primer
gagtcaggacctggcctcgtgaaacct

SEQ ID NO: 8 - DNA, 27 bp, Artificial
  Description of Artificial Sequence Primer
aggtttcacgaggccaggtcctgactc

SEQ ID NO: 9 - DNA, 27 bp, Artificial
  Description of Artificial Sequence Primer
tctgtcactggctactccatcaccagt

SEQ ID NO: 10 - DNA, 27 bp, Artificial
  Description of Artificial Sequence Primer
actggtgatggagtagccagtgacaga

SEQ ID NO: 11 - DNA, 27 bp, Artificial
  Description of Artificial Sequence Primer
aactggatccggcagttcccagggaat

SEQ ID NO: 12 - DNA, 27 bp, Artificial
  Description of Artificial Sequence Primer
attccctgggaactgccggatccagtt

SEQ ID NO: 13 - DNA, 33 bp, Artificial
  Description of Artificial Sequence Primer
gggaataaacttgaatggatgggttacataagc

SEQ ID NO: 14 - DNA, 33 bp, Artificial
  Description of Artificial Sequence Primer
gcttatgtaacccatccattcaagtttattccc

SEQ ID NO: 15 - DNA, 27 bp, Artificial
  Description of Artificial Sequence Primer
tccaagaaccaagcctacctccagttg

SEQ ID NO: 16 - DNA, 27 bp, Artificial
  Description of Artificial Sequence Primer
caactggaggtaggcttggttcttgga

SEQ ID NO: 17 - DNA, 27 bp, Artificial
  Description of Artificial Sequence Primer
ctccagttgaattctgtgactgctgag

SEQ ID NO: 18 - DNA, 27 bp, Artificial
  Description of Artificial Sequence Primer
ctcagcagtcacagaattcaactggag

SEQ ID NO: 19 - DNA, 27 bp, Artificial
  Description of Artificial Sequence Primer
acagccacatattactgtgcaagaagg

SEQ ID NO: 20 - DNA, 27 bp, Artificial
  Description of Artificial Sequence Primer
ccttcttgcacagtaatatgtggctgt

SEQ ID NO: 21 - DNA, 27 bp, Artificial
  Description of Artificial Sequence Primer
ggatcgagatctcgatcccgcgaaatt

SEQ ID NO: 22 - DNA, 30 bp, Artificial
  Description of Artificial Sequence Primer
gcctggcttctgcaggtaccaatgtaaata

SEQ ID NO: 23 - protein, 6 aa, Artificial
  Description of Artificial Sequence Linker Protein
MHGTKL

(19) United States
(12) Patent Application Publication
(10) Pub. No.: US 2006/0160174 A1
(43) Pub. Date: Jul. 20, 2006

(54) Modified l49-sfv exhibiting increased stability and methods of use thereof

(76) Inventors: Charlotte F McDonagh, Seatle, WA (US); Joseph A Francisco, Woodway, WA (US)

Correspondence Address: JONES DAY, 222 EAST 41ST ST, NEW YORK, NY, 10017, US

(21) Appl. No.: 10/537,143

(22) PCT Filed: Dec. 2, 2002
(86) PCT No.: PCT/US02/38414

§ 371 Date: Oct. 24, 2005

(51) Int. Cl. C07K 16/44; C07H 21/04; C12P 21/06; C12N 5/06

(52) U.S. Cl. 435/69.1; 435/320.1; 435/326; 530/388.15; 536/23.53

(57) ABSTRACT

The present invention relates to a modified L49 single chain antibody (L49-sFv) that exhibits increased refolding efficiency and/or greater stability in mouse serum, and surprisingly substantially maintains binding affinity for its binding ligand, p97 melanotransferrin. p97 melanotransferrin is expressed on the surface of a number of types of cancer (carcinoma) cells, e.g., melanoma cells, lung cancer cells, renal cancer cells, colon cancer cells. The present invention also relates to a modified L49-sFv fused or conjugated to a therapeutic agent, such as a cytotoxic molecule or a pro-drug converting enzyme. The present invention also relates to methods of using the modified L49-sFv molecules fused or conjugated to a therapeutic agent for treatment and/or prophylaxis of cancer, which cancer cells express p97 melanotransferrin.

### 1. FIELD OF THE INVENTION

The present invention relates to a modified L49 single chain antibody (L49-sFv) that exhibits increased refolding efficiency and/or greater stability in mouse serum, and surprisingly substantially maintains binding affinity for its binding ligand, p97 melanotransferrin. p97 melanotransferrin is expressed on the surface of a number of types of cancer (carcinoma) cells, e.g., melanoma cells, lung cancer cells, renal cancer cells, colon cancer cells. The present invention also relates to a modified L49-sFv fused or conjugated to a therapeutic agent, such as a cytotoxic molecule or a pro-drug converting enzyme. The present invention also relates to methods of using the modified L49-sFv molecules fused or conjugated to a therapeutic agent for treatment and/or prophylaxis of cancer, which cancer cells express p97 melanotransferrin.

### 2. BACKGROUND OF THE INVENTION

Antibody-directed enzyme prodrug therapy (ADEPT) is a two-step approach to cancer therapy in which monoclonal antibodies (mAbs) are used for the delivery of enzymes to tumor-cell surfaces. The localized enzymes are able to activate subsequently administered prodrugs into active anticancer agents, which can then penetrate into the cells and exert cytotoxic activities (Senter and Springer, 2001, Adv. Drug Deliv. Rev. 53, 247-2641). L49-sFv-bL is a recombinant fusion protein composed of a single-chain Fv derived from the L49 mAb that binds to the p97 melanotransferrin antigen expressed by human melanomas and some carcinomas (Siemers et al., 1997, Bioconjug Chem. 8, 510-5192) and a mutated form of the Enterobacter cloacae β-lactamase (bL) (Siemers et al., 1996, Biochemistry 35, 2104-21113). The β-lactamase enzyme rapidly catalyzes the hydrolysis of cephalosporin-containing prodrugs and has been used for the release of such drugs as melphalan (Kerr et al., 1998, Bioconjug Chem. 9, 255-2594), nitrogen mustards (Vrudhula et al., 1993, Bioconjug Chem. 4, 334-3405), vinca derivatives (Meyer et al., 1995, Bioconjug Chem. 6, 440-446), paclitaxel (Rodrigues et al., 1995, Chem. Biol. 2, 223-227), doxorubicin (Vrudhula et al., 1995, J Med. Chem. 38, 1380-1385) and mitomycin C (Vrudhula et al., 1997, J Med. Chem. 40, 2788-2792). In vivo therapy experiments in mouse models have demonstrated the effectiveness of this combination for treatment of melanomas (Kerr et al., 1998, Bioconjug Chem. 9, 255-2594) and lung adenocarcinomas (Kerr et al., 1999, Bioconjug. Chem. 10, 1084-1089).

Previous studies utilized L49-sFv-bL solubly expressed in E. coli and purified from lysed cells using two affinity steps (Siemers et al., 1997, Bioconjug. Chem. 8, 510-519). While this allowed for the generation of modest amounts (1-4 mg/liter) of purified protein from shake-flasks and small fermenters (Id), it did not provide enough material for clinical development. To address this, the present inventors explored the large-scale production of L49-sFv-bL from refolded E. coli inclusion bodies, but encountered significant problems since the protein did not refold efficiently. Others have also reported difficulties in refolding scFv molecules (Worn and Pluckthun, 2001, J. Mol. Biol. 305, 989-1010).

Several rational engineering methods have been used to improve the refolding properties of problematic scFv fragments (Worn and Pluckthun, 2001, J. Mol. Biol. 305,989-1010). One possibility is to graft the CDRs onto the framework of a more stable scFv (Jung and Pluckthun, 1997, Protein Eng 10, 959-966; Willuda et al., 1999, Cancer Res. 59, 5758-5767). However, CDR grafting has been shown to reduce the binding affinity of the scFv (Worn and Pluckthun, 1999, Biochemistry 38, 8739-8750). Another method has been to compare the framework of the unstable scFv with that of a homologous stable scFv and make the appropriate changes (Knappik and Pluckthun, 1995, Protein Eng 8, 81-89). Of course, this approach is only possible if a homologous stable scFv can be identified, which was not the case for L49-sFv. Introduction of an interface disulfide bond between the V_{H} and V_{L} domain has also been used to stabilize scFv fragments (Glockshuber et al., 1990, Biochemistry 29, 1362-1367). However, the introduction of extra cysteines into the scFv may further complicate refolding by allowing additional incorrect intra-molecular disulfide bonds to form.

Sequence statistics have also been used to identify problematic amino acids in scFv frameworks. A technique, used successfully to engineer a scFv immunotoxin, has been to identify ‘unusual’ amino acids in the scFv frameworks by aligning the target sequence with known stable variable domains and scanning all residues for deviations from the Kabat consensus. ‘Rare’ residues with less than 5% frequency in the Kabat database, that were also incompatible with their solvent exposure state, were mutated to the consensus residue. Up to 18-fold increases in yield were observed although binding affinity was compromised 2-3.5-fold (Chowdhury et al., 1998, J. Mol. Biol. 281, 917-928). This technique provides a straightforward initial screen to identify potential ‘problem’ amino acids within an scFv framework. Alignment of scFvs to their general subgroup consensus sequences has also revealed problematic framework amino acids. Stabilizing mutations have been introduced in both V_{H} and V_{L} domains using this approach (Steipe et al., 1994, J Mol Biol. 240, 188-192; Wirtz and Steipe, 1999, Protein Sci. 8, 2245-2250). For Kappa V_{L} domains, this strategy was effective for approximately 60% of mutations (Steipe et al., 1994, J Mol Biol. 240, 188-192).

Thus, despite the current state of the technology, there remains a need in the art for a modified L49-sFv single-chain antibody that exhibits greater stability and/or refolding efficiency and substantially maintains binding affinity for its ligand as compared to the parental single-chain antibody.

Citation of a reference in this or any section of the specification shall not be construed as an admission that such reference is prior art to the present invention.

### 3. SUMMARY OF THE INVENTION

The present invention is directed to proteins comprising modified L49 single-chain antibodies (L49-sFv) and methods for their use in treating or preventing cancer, wherein the cancer cells express p97 melanotransferrin, the binding ligand of L49-sFv. In one embodiment of the invention, the modified L49-sFv comprises an amino acid substitution at position 85 (Kabat position H82B) of phenylalanine for serine, threonine or alanine of SEQ ID NO:2. In another embodiment of the invention, the modified L49-sFv comprises an amino acid substitution at position 95 (Kabat position H91) of asparagine for tyrosine or phenylalanine of SEQ ID NO:2. In yet another embodiment, the modified L49-sFv comprises an amino acid substitution at position 85 (Kabat position H82B) of phenylalanine for serine, threonine or alanine, and at position 95 (Kabat position H91) of asparagine for tyrosine or phenylalanine of SEQ ID NO:2.

In another embodiment, the modified L49-sFv comprises an amino acid substitution at position 40 (Kabat position H39) of lysine for glutamine, and at position 95 (Kabat position H91) of asparagine for tyrosine or phenylalanine of SEQ ID NO:2. In yet another embodiment, the modified L49-sFv comprises an amino acid substitution at position 40 (Kabat position H39) of lysine for glutamine, and at position 85 (Kabat position H82B) of phenylalanine for serine, threonine or alanine of SEQ ID NO:2. In another embodiment, the modified L49-sFv comprises an amino acid substitution at position 40 (Kabat position H39) of lysine for glutamine, at position 85 (Kabat position H82B) of phenylalanine for serine, threonine or alanine and at position 95 (Kabat position H91) of asparagine for tyrosine or phenylalanine of SEQ ID NO:2.

The modified L49 single-chain antibodies of the present invention exhibit increased refolding efficiency and/or greater stability in mouse serum, and surprisingly substantially maintain binding affinity for its binding ligand, p97 melanotransferrin. As used herein, substantially maintain binding affinity means that the modified L49-sFv has at least 75%, 85%, 90%, 95% or 99% of the binding affinity of the parental L49-sFv, or has a binding affinity that is equal to or greater than the parental L49-sFv.

In certain embodiments of the invention, the modified L49-sFv is fused or conjugated to a therapeutic agent, such as a cytotoxic molecule or a pro-drug converting enzyme. In an aspect of this embodiment, the therapeutic agent is fused via a peptide bond to said modified L49-sFv at either the N-terminus or C-terminus of said modified L49-sFv. In one embodiment, the therapeutic agent is the pro-drug converting enzyme β-lactamase which is fused via peptide bond to the N-terminus of the modified L49-sFv. In a preferred embodiment, the modified L49-sFv comprises an amino acid substitution at position 40 (Kabat position H39) of lysine for glutamine, at position 85 (Kabat position H82B) of phenylalanine for serine, and at position 95 (Kabat position H91) of asparagine for tyrosine of SEQ ID NO:2, and is fused to a therapeutic agent at the N-terminus of said L49-sFv.

The present invention is also directed to a nucleic acid comprising a nucleotide sequence encoding a modified L49-sFv, or a modified L49-sFv fusion protein. In a particular embodiment, the nucleic acid comprises the nucleotide sequence of SEQ ID NO:1 in which nucleotide residues 253-255 are changed to AGT, AGC, TCT, TCC, TCA, TCG, ACC, ACA, ACG, ACT, GCC, GCA, GCG or GCT; the nucleotide sequence of SEQ ID NO:1 in which nucleotide residues 283-285 are changed to TAC, TAT, TTC or TTT; the nucleotide sequence of SEQ ID NO:1 in which nucleotide residues 253-255 are changed to AGT, AGC, TCT, TCC, TCA, TCG, ACC, ACA, ACG, ACT, GCC, GCA, GCG or GCT, and nucleotide residues 283-285 are changed to TAC, TAT, TTC or TTT; the nucleotide sequence of SEQ ID NO:1 in which nucleotide residues 118-120 are changed to CAG or CAA, and nucleotide residues 283-285 are changed to TAC, TAT, TTC or TTT; the nucleotide sequence of SEQ ID NO:1 in which nucleotide residues 118-120 are changed to CAG or CAA, and nucleotide residues 253-255 are changed to AGT, AGC, TCT, TCC, TCA, TCG, ACC, ACA, ACG, ACT, GCC, GCA, GCG or GCT; and the nucleotide sequence of SEQ ID NO:1 in which nucleotide residues 118-120 are changed to CAG or CAA, nucleotide residues 253-255 are changed to AGT, AGC, TCT, TCC, TCA, TCG, ACC, ACA, ACG, ACT, GCC, GCA, GCG or GCT, and nucleotide residues 283-285 are changed to TAC, TAT, TTC or TTT.

The present invention is also directed to a molecule comprising a modified L49-sFv, fusion protein or conjugate thereof, as well as to a molecule comprising a nucleic acid encoding the modified L49-sFv, fusion protein or conjugate thereof.

The present invention is also directed to a recombinant vector comprising a nucleic acid encoding a modified L49-sFv, or fusion protein thereof, operably linked to a promoter, and host cells comprising said vector. In another embodiment, the present invention is directed to a method for producing a modified L49-sFv protein, or fusion protein thereof, comprising culturing a host cell of the invention such that the nucleic acid is expressed by the cell to produce its encoded modified L49-sFv protein; and isolating the expressed protein.

The present invention is also directed to a pharmaceutical composition comprising a purified modified L49-sFv of the invention or its encoding nucleic acid, and a pharmaceutically acceptable carrier. As used herein, the term “purified” means that the product is substantially free of other biological material with which it is naturally associated, or free from other biological materials derived, for example, from a recombinant host cell that has been genetically engineered to express the polypeptide of the invention. That is, a purified, modified L49-sFv is at least 70-95% pure L49-sFv by weight, preferably at least 75% pure L49-sFv by weight, and most preferably at least 95% pure L49-sFv by weight, or most preferably 98% pure L49-sFv by weight.

The present invention is also directed to a method for treating or preventing cancer, wherein the cancer expresses p97 melanotransferrin comprising administering to a subject in need of such treatment or prevention, an effective amount of a pharmaceutical composition comprising a modified L49-sFv fused or conjugated to a therapeutic agent, or a nucleic acid encoding said modified L49-sFv fused to a therapeutic agent. Exemplary cancers that express p97 melanotransferrin include, but are not limited to, melanoma, breast cancer, lung cancer, renal cancer, ovarian cancer and colon cancer. In certain embodiments where the therapeutic agent is a pro-drug converting enzyme, the method further comprises administering the appropriate pro-drug, i.e., the pro-drug that is converted to its active form by said pro-drug converting enzyme, either before, concurrently with, or after administration of the modified L49-sFv.

The present invention may be understood more fully by reference to the following detailed description, illustrative examples of specific embodiments and the appended figures.

### 4. BRIEF DESCRIPTION OF THE DRAWINGS

FIGS. 1A-1D. FIGS. 1A-1B depicts the nucleotide sequence (SEQ ID NO:1) (FIG. 1A) and its encoded amino acid sequence (SEQ ID NO:2) (FIG. 1B) of the parental L49-sFv molecule. FIG. 1C depicts the nucleotide sequence (SEQ ID NO:3) of the parental L49-sFv-bL molecule and FIG. 1D depicts the amino acid sequence of the parental L49-sFv-bL (SEQ ID NO:4), including the wild type heavy and light chain variable regions, the linker separating the chains and the P-lactamase sequence. The asterisks (*) indicate the residues that are mutated to provide the novel modified L49-sFv molecules of the present invention.

FIGS. 2A-2B depict the refolding efficiency of the modified L49-sFv-bL fusion proteins as measured by binding of refolded molecules to p97 melanotransferrin. The more efficient the refolding, the higher the amount of binding to p97. FIG. 2A is a graph showing the results of binding of refolded mutants FP930 (K H39 Q), FP935 (Y H47 W), FP940 (Y H78 A), FP945 (F H82B S), FP950 (N H91 Y), FP960 (F H82B S, N H91 Y), FP965 (K H39 Q, N H91 Y) and parental protein FP95 to antigen p97 using a solid phase binding assay. FIG. 2B is a graph showing the results of binding of refolded triple mutant FP990 (K H39 Q, F H82B S, N H91 Y) compared to binding of double mutant FP965 (K H39 Q, N H91 Y) and parental protein FP95. All experiments were performed in duplicate. For additional details, see Section 6.2.2.

FIGS. 3A and 3B show western blot analyses of refolded molecules, wherein 10 mmoles of FP950 (N H91 Y), FP960 (F H82B S, N H91 Y), FP965 (K H39 Q, N H91 Y), FP990 (K H39 Q, F H82B S, N H91 Y) and parental protein FP95 and sL49-sFv-bL were separated as indicated on a 10% TrislGlycine gel under non-reducing conditions (FIG. 3A) or under reducing conditions (FIG. 3B). Proteins were transferred onto nitrocellulose and blotted with rabbit polyclonal anti-β-lactamase antibody followed by incubation with HRP-conjugated secondary antibody. Blots were developed by addition of colorimetric substrate DAB. For additional details, see Section 6.2.3

FIG. 4 shows an SDS-PAGE analysis under non-reducing conditions on a 10% Tris/Glycine gel of FP95 (parental), FP999 (K H39 Q, F H82B S, N H91 Y, bL-L49-scFv orientation), FP960 (F H82B S, N H91 Y), FP965 (K H39 Q, N H91 Y) and FP990 (K H39 Q, F H82B S, N H91 Y) proteins following ion exchange chromatography. The additional bands observed in the FP95 (parental) sample are degradation products. For additional details, see Section 6.2.4.

FIGS. 5A-5E are graphs showing the stability of L49-sFv-bL parental and mutant molecules in mouse plasma. Proteins from each time point, at indicated concentrations, were incubated in wells coated with p97 and following wash steps protein binding was determined using β-lactamase substrate nitrocefin. All experiments were performed in duplicate. For additional details, see Section 6.2.6.

FIG. 6 is a graph depicting the cytotoxic effects of parental FP95 and mutant molecules, FP990 (K H39 Q, F H82B S, N H91 Y) and FP999 (K H39 Q, F H82B S, N H91 Y, bL-L49-scFv orientation), in combination with C-Mel on H3677 melanoma cells as determined by the redox indicator alamarBlue™. Each point was recorded in quadruplet. The effects were compared to cells treated with pro-drug C-Mel or melphalan (Mel) alone. For additional details, see Section 6.2.7.

FIG. 7 is a graph depicting the in vivo therapeutic effects of L49-sFv-bL mutants FP990 (K H39 Q, F H82B S, N H91 Y) and FP999 (K H39 Q, F H82B S, N H91 Y, bL-L49-sFv orientation) in combination with C-Mel. Nude mice (five mice per group) were treated with L49-sFv-bL (FP990 or FP999) at 1 mg/kg followed 24 hours later by C-Mel. The average tumor volumes were plotted until one or more mice were removed from the experiment due to tumor outgrowth. C-Mel was also injected without prior treatment with L49-sFv-bL. For additional details, see Section 6.2.8.

### 5. DETAILED DESCRIPTION OF THE INVENTION

The present invention is directed to modified L49 single-chain antibodies (modified L49-sFv). The modified L49 single-chain antibodies of the present invention exhibit increased refolding efficiency and/or greater stability in mouse serum, and surprisingly, substantially maintain binding affinity for its binding ligand, p97 melanotransferrin.

In certain embodiments of the invention, the modified L49-sFv is fused or conjugated to therapeutic agent, such as a cytotoxic molecule or a pro-drug converting enzyme. In an aspect of this embodiment, the therapeutic agent is fused via a peptide bond to said modified L49 sFv at either the N-terminus or C-terminus of said modified L49 sFv. In a preferred embodiment, the therapeutic agent is pro-drug converting enzyme beta-lactamase which is fused via peptide bond to the N-terminus of the modified L49-sFv.

The present invention is also directed to a nucleic acid comprising a nucleotide sequence encoding a modified L49 sFv, or modified L49-sFv fused to a therapeutic agent (L49-sFv fusion protein). The present invention is also directed to a recombinant vector comprising a nucleic acid encoding a modified L49-sFv or fusion protein thereof operably linked to a promoter, and host cells comprising said vector. In another embodiment, the present invention is directed to a method for producing a modified L49 sFv protein or L49-sFv fusion protein comprising culturing a host cell of the invention such that the nucleic acid is expressed by the cell to produce its encoded modified L49 sFv protein or fusion protein; and isolating the expressed protein or fusion protein.

The present invention is also directed to a pharmaceutical composition comprising a purified modified L49-sFv of the invention or a purified nucleic acid encoding said modified L49-sFv, and a pharmaceutically acceptable carrier.

The present invention is also directed to a method for treating or preventing cancer, wherein the cancer expresses p97 melanotransferrin comprising administering to a subject in need of such treatment or prevention, an effective amount of a pharmaceutical composition comprising a modified L49-sFv fused or conjugated to a therapeutic agent, or a purified nucleic acid encoding said modified L49-sFv. Exemplary cancer cells that express p97 melanotransferrin include, but are not limited to, melanoma and certain breast, lung, ovarian, renal and colon cancers. In certain embodiments where the therapeutic agent is a pro-drug converting enzyme, the method further comprises administering the appropriate pro-drug, either before, concurrently with, or after administration of the modified L49-sFv. The appropriate pro-drug is one that is acted upon or converted by the pro-drug converting enzyme to its active form.

For clarity of disclosure, and not by way of limitation, the detailed description of the invention is divided into the subsections which follow.

5.1 Proteins of the Invention and Methods for their Production

The present invention encompasses proteins that comprise a modified L49 single chain antibody (L49-sFv), which modified L49-sFv exhibits increased refolding efficiency and/or greater stability in mouse serum, and surprisingly, substantially maintains binding affinity for its binding ligand, p97 melanotransferrin. In an embodiment of the invention, the modification of L49-sFv comprises an amino acid substitution at position 85 (Kabat position H82B) of phenylalanine for serine, threonine or alanine of SEQ ID NO:2; or an amino acid substitution at position 95 (Kabat position H91) of asparagine for tyrosine or phenylalanine of SEQ ID NO:2. In another embodiment, the modification comprises an amino acid substitution at position 85 (Kabat position H82B) of phenylalanine for serine, threonine or alanine, and at position 95 (Kabat position H91) of asparagine for tyrosine or phenylalanine of SEQ ID NO:2; or an amino acid substitution at position 40 (Kabat position H39) of lysine for glutamine, and at position 95 (Kabat position H91) of asparagine for tyrosine or phenylalanine of SEQ ID NO:2; or an amino acid substitution at position 40 (Kabat position H39) of lysine for glutamine, and at position 85 (Kabat position H82B) of phenylalanine for serine, threonine or alanine of SEQ ID NO:2. In yet another embodiment, the modification comprises an amino acid substitution at position 40 (Kabat position H39) of lysine for glutamine, at position 85 (Kabat position H82B) of phenylalanine for serine, threonine or alanine and at position 95 (Kabat position H91) of asparagine for tyrosine or phenylalanine of SEQ ID NO:2.

In one embodiment of the invention, a modified L49-sFv of the invention is fused to a marker sequence, such as a peptide, to facilitate purification. In preferred embodiments, the marker amino acid sequence is a hexa-histidine peptide, such as the tag provided in a pQE vector (QIAGEN, Inc., 9259 Eton Avenue, Chatsworth, Calif., 91311), among others, many of which are commercially available. As described in Gentz et al., 1989, Proc. Natl. Acad. Sci. USA 86:821-824, for instance, hexa-histidine provides for convenient purification of the fusion protein. Other peptide tags useful for purification include, but are not limited to, the “HA” tag, which corresponds to an epitope derived from the influenza hemagglutinin protein (Wilson et al., 1984, Cell 37,767) and the “flag” tag. Such fusion proteins can be generated by standard recombinant methods known to those of skill in the art.

In another embodiment, the present invention is directed to a fusion protein comprising a modified L49-sFv of the invention fused via a peptide bond to a therapeutic agent. For example, a modified L49-sFv protein of the invention may be fused via a peptide bond to a pro-drug converting enzyme, or to a cytotoxic agent, such as a chemotherapeutic agent, a toxin (e.g., a cytostatic or cytocidal agent). Thus, a nucleic acid of the invention (encoding a modified L49-sFv) may be modified to functionally couple the coding sequence of a pro-drug converting enzyme with the coding sequence of the modified L49-sFv of the invention, such that a fusion protein comprising the functionally active pro-drug converting enzyme and the modified L49-sFv of the invention is expressed in the subject upon administration of the nucleic acid in accordance with the gene therapy methods described in Section 5.3, infra.

Exemplary nucleic acids of the invention that encode a modified L49-sFv include those that comprise the nucleotide sequence of SEQ ID NO:1 in which nucleotide residues 253-255 are changed to AGT, AGC, TCT, TCC, TCA, TCG, ACC, ACA, ACG, ACT, GCC, GCA, GCG or GCT; the nucleotide sequence of SEQ ID NO:1 in which nucleotide residues 283-285 are changed to TAC, TAT, TTC or TTT; the nucleotide sequence of SEQ ID NO:1 in which nucleotide residues 253-255 are changed to AGT, AGC, TCT, TCC, TCA, TCG, ACC, ACA, ACG, ACT, GCC, GCA, GCG or GCT, and nucleotide residues 283-285 are changed to TAC, TAT, TTC or TTT; the nucleotide sequence of SEQ ID NO:1 in which nucleotide residues 118-120 are changed to CAG or CAA, and nucleotide residues 283-285 are changed to TAC, TAT, TTC or TTT; the nucleotide sequence of SEQ ID NO:1 in which nucleotide residues 118-120 are changed to CAG or CAA, and nucleotide residues 253-255 are changed to AGT, AGC, TCT, TCC, TCA, TCG, ACC, ACA, ACG, ACT, GCC, GCA, GCG or GCT; and the nucleotide sequence of SEQ ID NO:1 in which nucleotide residues 118-120 are changed to CAG or CAA, nucleotide residues 253-255 are changed to AGT, AGC, TCT, TCC, TCA, TCG, ACC, ACA, ACG, ACT, GCC, GCA, GCG or GCT, and nucleotide residues 283-285 are changed to TAC, TAT, TTC or TTT.

Further, a nucleic acid of the invention may be modified to functionally couple the coding sequence of a cellular toxin with the coding sequence of a modified L49-sFv of the invention, such that a fusion protein comprising the cellular toxin and the modified L49-sFv of the invention is expressed in the subject upon administration of the nucleic acid in accordance with the gene therapy methods described in Section 5.3, infra.

Prodrug converting enzymes are widely employed for use in gene therapy of malignant cancers (Vile and Hart, 1993, Cancer Res. 53, 3860-3864; Moolten and Wells, 1990, J. Natl. Cancer Inst. 82, 297-300; Wagner, et al., 1981, Proc. Natl. Acad. Sci. USA 78, 1441-1445; Mullen, 1994, Cancer Res. 54, 1503-1506; Huber et al., 1993, Cancer Res. 53, 4619-4625; Waldman et al., 1983, J. Biol. Chem. 258, 11571-11575; Mullen, et al., 1992, Proc. Natl. Acad. Sci. 89, 33-37; Austin and Huber, 1993, Mol. Pharmacol. 43, 380-387). Illustrative examples of pro-drug converting enzymes are listed on page 33 and in Table 2 of International Patent Publication WO 96/40238 by Pawelek et al., which is incorporated herein in its entirety. Exemplary pro-drug converting enzymes include Herpes simplex thymidine kinase (TK) and bacterial cytosine deaminase (CD). TK phosphorylates the non-toxic substrates acyclovir and ganciclovir, rendering them toxic via their incorporation into genomic DNA. CD converts the non-toxic 5-fluorocytosine (5-FC) into 5-fluorouracil (5-FU), which is toxic via its incorporation into RNA. Additional examples of pro-drug converting enzymes encompassed by the present invention include cytochrome p450 NADPH oxidoreductase, which acts upon mitomycin C, and porfiromycin (Murray et al., 1994, J. Pharmacol. Exp. Therapeut. 270, 645-649). Additional specific exemplary pro-drug converting enzymes are alkaline phosphatase, α-galactosidase, β-galactosidase, aminopeptidase, aryl sulfatase, glucose oxidase, caspase, carboxylesterase, xanthine oxidase, elastase, nitroreductase, carboxypeptidase G2, beta-glucuronidase, penicillin-V-amidase, penicillin-G-amidase, β-lactamase, β-glucosidase, and carboxypeptidase A. In a preferred embodiment of the present invention, the pro-drug converting enzyme is Enterobacter cloacae β-lactamase. Illustrative examples of a cytotoxic agent include, but are not limited to, abrin, ricin A, bryodin, pseudomonas exotoxin, diphtheria toxin, saporin and a porin protein.

The modified L49 sFv proteins and fusion proteins of the invention can be produced by any method known in the art for the synthesis of proteins, in particular, by chemical synthesis or preferably, by recombinant expression techniques. Further, the fusion does not necessarily need to be direct, but may occur through linker sequences.

Recombinant expression of a L49-sFv protein or fusion protein of the invention requires construction of an expression vector containing a nucleic acid that encodes the protein. Once a nucleic acid encoding a protein of the invention has been obtained, the vector for the production of the protein molecule may be produced by recombinant DNA technology using techniques well known in the art. Thus, methods for preparing a protein by expressing a nucleic acid containing nucleotide sequence encoding said protein are described herein. Methods which are well known to those skilled in the art can be used to construct expression vectors containing coding sequences and appropriate transcriptional and translational control signals. These methods include, for example, in vitro recombinant DNA techniques, synthetic techniques, and in vivo genetic recombination. The invention, thus, provides replicable vectors comprising a nucleotide sequence encoding a protein of the invention operably linked to a promoter.

In a preferred embodiment of the present invention, an expression vector is constructed comprising a nucleotide sequence encoding a modified L49-sFv protein or fusion protein of the present invention allowing for expression of a modified L49-sFv protein or fusion protein of the invention. In another preferred embodiment, the vector comprises a nucleotide sequence encoding a modified L49-sFv-β-lactamase fusion protein operably linked to a promoter.

The expression vector is transferred to a host cell by conventional techniques and the transfected cells are then cultured by conventional techniques to produce a protein of the invention. Thus, the invention encompasses host cells containing a nucleic acid encoding a protein of the invention, operably linked to a heterologous promoter. In preferred embodiments for the expression of single-chained antibodies, a vector encoding both variable regions may be expressed in the host cell for expression of the single chain immunoglobulin molecule (scFv), as detailed below.

A variety of host-expression vector systems may be utilized to express the proteins molecules of the invention. Such host-expression systems represent vehicles by which the coding sequences of interest may be produced and subsequently purified, but also represent cells which may, when transformed or transfected with the appropriate nucleotide coding sequences, express a protein of the invention in situ. These include but are not limited to microorganisms such as bacteria (e.g., E. coli, B. subtilis) transformed with recombinant bacteriophage DNA, plasmid DNA or cosmid DNA expression vectors containing antibody coding sequences; yeast (e.g., Saccharomyces, Pichia) transformed with recombinant yeast expression vectors containing antibody coding sequences; insect cell systems infected with recombinant virus expression vectors (e.g., baculovirus) containing antibody coding sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing antibody coding sequences; or mammalian cell systems (e.g., COS, CHO, BHK, 293, 3T3 cells) harboring recombinant expression constructs containing promoters derived from the genome of mammalian cells (e.g., metallothionein promoter) or from mammalian viruses (e.g., the adenovirus late promoter; the vaccinia virus 7.5K promoter). Preferably, bacterial cells such as Escherichia coli, and more preferably, eukaryotic cells, are used for the expression of a recombinant protein of the invention. For example, mammalian cells such as Chinese hamster ovary cells (CHO), in conjunction with a vector such as the major. intermediate early gene promoter element from human cytomegalovirus is an effective expression system for proteins of the invention (Foecking et al., 1986, Gene 45, 101; Cockett et al., 1990, Bio/Technology 8, 2).

In bacterial systems, a number of expression vectors may be advantageously selected depending upon the use intended for the folding and post-translation modification requirements protein being expressed. Where possible, when a large quantity of such a protein is to be produced, for the generation of pharmaceutical compositions comprising a protein of the invention, vectors which direct the expression of high levels of fusion protein products that are readily purified may be desirable. Such vectors include, but are not limited to, the E. coli pET vector system from Novagen (Madison, Wis.) which uses a T7 promoter, the E. coli expression vector pUR278 (Ruther et al, 1983, EMBO J. 2, 1791), in which the antibody coding sequence may be ligated individually into the vector in frame with the lac Z coding region so that a fusion protein is produced; pIN vectors (Inouye & Inouye, 1985, Nucleic Acids Res. 13, 3101-3109; Van Heeke & Schuster, 1989, J. Biol. Chem. 24, 5503-5509); and the like. pGEX vectors may also be used to express fusion proteins with glutathione S-transferase (GST). In general, such fusion proteins are soluble and can easily be purified from lysed cells by adsorption and binding to matrix glutathione-agarose beads followed by elution in the presence of free glutathione. The pGEX vectors are designed to include thrombin or factor Xa protease cleavage sites so that the cloned target gene product can be released from the GST moiety.

In an insect system, Autographa californica nuclear polyhedrosis virus (AcNPV) is used as a vector to express foreign genes. The virus grows in Spodoptera frugiperda cells. The antibody coding sequence may be cloned individually into non-essential regions (for example the polyhedrin gene) of the virus and placed under control of an AcNPV promoter (for example the polyhedrin promoter).

In mammalian host cells, a number of viral-based expression systems may be utilized. In cases where an adenovirus is used as an expression vector, the coding sequence of the protein of the invention may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing the protein of the invention in infected hosts. (See, e.g., Logan & Shenk, 1984, Proc. Natl. Acad. Sci. USA 81, 355-359). Specific initiation signals may also be required for efficient translation of inserted coding sequences. These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (Bittner et al., 1987, Methods in Enzymol. 153, 51-544).

In addition, a host cell strain may be chosen which modulates the expression of the inserted sequences, or modifies and processes the gene product in the specific fashion desired. Such modifications (e.g., glycosylation) and processing (e.g., cleavage) of protein products may be important for the function of the protein of the invention. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells which possess the cellular machinery for proper processing of the primary transcript, glycosylation, and phosphorylation of the gene product may be used. Such mammalian host cells include but are not limited to CHO, VERO, BHK, Hela, COS, MDCK, 293, 3T3, and W138.

For long-term, high-yield production of recombinant proteins, stable expression is preferred. For example, cell lines which stably express the protein of the invention may be engineered. Rather than using expression vectors which contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method may advantageously be used to engineer cell lines which express the protein of the invention.

A number of selection systems may be used, including but not limited to the herpes simplex virus thymidine kinase (Wigler et al., 1977, Cell 11, 223), hypoxanthine-guanine phosphoribosyltransferase (Szybalska & Szybalski, 1992, Proc. Natl. Acad. Sci. USA 48, 202), and adenine phosphoribosyltransferase (Lowy et al., 1980, Cell 22, 8-17) genes can be employed in tk-, hgprt- or aprt-cells, respectively. Also, antimetabolite resistance can be used as the basis of selection for the following genes: dhfr, which confers resistance to methotrexate (Wigler et al., 1980, Proc. Natl. Acad. Sci. USA 77, 357; O'Hare et al., 1981, Proc. Natl. Acad. Sci. USA 78, 1527); gpt, which confers resistance to mycophenolic acid (Mulligan & Berg, 1981, Proc. Natl. Acad. Sci. USA 78, 2072); neo, which confers resistance to the aminoglycoside G418 (Clinical Pharmacy 12:488-505; Wu and Wu, 1991, Biotherapy 3, 87-95 ; Tolstoshev, 1993, Ann. Rev. Pharmacol. Toxicol. 32, 573-596; Mulligan, 1993, Science 260, 926-932 ; and Morgan and Anderson, 1993, Ann. Rev. Biochem. 62, 191-217; May, 1993, TIB TECH 11(5), 155-215); and hygro, which confers resistance to hygromycin (Santerre et al., 1984, Gene 30, 147). Methods commonly known in the art of recombinant DNA technology may be routinely applied to select the desired recombinant clone, and such methods are described, for example, in Ausubel et al. (eds.), Current Protocols in Molecular Biology, John Wiley & Sons, NY (1993); Kriegler, Gene Transfer and Expression, A Laboratory Manual, Stockton Press, NY (1990); and in Chapters 12 and 13, Dracopoli et al. (eds), Current Protocols in Human Genetics, John Wiley & Sons, NY (1994); Colberre-Garapin et al., 1981, J. Mol. Biol 150, 1, which are incorporated by reference herein in their entireties.

The expression levels of a protein of the invention can be increased by vector amplification (for a review, see Bebbington and Hentschel, The Use of Vectors Based on Gene Amplification for the Expression of Cloned Genes in Mammalian Cells in DNY Cloning, Vol. 3. (Academic Press, New York, 1987)). When a marker in the vector system expressing antibody is amplifiable, increase in the level of inhibitor present in culture of host cell will increase the number of copies of the marker gene. Since the amplified region is associated with the antibody gene, production of the protein of the invention will also increase (Crouse et al., 1983, Mol. Cell. Biol. 3, 257).

Once a protein molecule of the invention has been produced by an animal, chemically synthesized, or recombinantly expressed, it may be purified by any method known in the art for purification of proteins, for example, by chromatography (e.g., ion exchange; affinity, particularly by affinity for the specific antigen, Protein A (for antibody molecules, or affinity for a heterologous fusion partner wherein the protein is a fusion protein; and sizing column chromatography), centrifugation, differential solubility, or by any other standard technique for the purification of proteins.

5.2 Conjugates

In yet another embodiment of the present invention, the proteins of the invention encompass modified L49-sFv proteins and fusion proteins that are conjugated to a heterologous protein, or to a therapeutic agent such as a cytotoxic agent or a pro-drug converting enzyme. Particularly suitable moieties for conjugation to proteins of the invention are chemotherapeutic agents, pro-drug converting enzymes, radioactive isotopes or a radionuclide (e.g., alpha-emitters such as, for example, ²¹²Bi, ²¹¹At, or beta-emitters such as, for example, ¹³¹I, ⁹⁰Y, or ⁶⁷Cu) or compounds, or toxins, e.g., a cytostatic or cytocidal agent). Further, the conjugation does not necessarily need to be direct, but may occur through linker sequences.

Drugs such as methotrexate (Endo et al., 1987, Cancer Research 47, 1076-1080), daunomycin (Gallego et al., 1984, Int. J. Cancer 33, 737-744), mitomycin C (MMC) (Ohkawa et al., 1986, Cancer Immunol. Immunother. 23, 81-86) and vinca alkaloids (Rowland et al., 1986, Cancer Immunol. Immunother. 21, 183-187) have been attached to antibodies and the derived conjugates have been investigated for anti-tumor activities. Care should be taken in the generation of chemotherapeutic agent conjugates to ensure that the activity of the drug and/or protein does not diminish as a result of the conjugation process.

Examples of chemotherapeutic agents include the following non-mutually exclusive classes of chemotherapeutic agents: alkylating agents, anthracyclines, antibiotics, antifolates, antimetabolites, antitubulin agents, auristatins, chemotherapy sensitizers, DNA minor groove binders, DNA replication inhibitors, duocarmycins, etoposides, fluorinated pyrimidines, lexitropsins, nitrosoureas, platinols, purine antimetabolites, puromycins, radiation sensitizers, steroids, taxanes, topoisomerase inhibitors, and vinca alkaloids.

Examples of individual chemotherapeutics that can be conjugated to a nucleic acid or protein of the invention include but are not limited to an androgen, anthramycin (AMC), asparaginase, 5-azacytidine, azathioprine, bleomycin, busulfan, buthionine sulfoximine, camptothecin, carboplatin, carmustine (BSNU), CC-1065, chlorambucil, cisplatin, colchicine, cyclophosphamide, cytarabine, cytidine arabinoside, cytochalasin B, dacarbazine, dactinomycin (formerly actinomycin), daunorubicin, decarbazine, docetaxel, doxorubicin, an estrogen, 5-fluordeoxyuridine, 5-fluorouracil, gramicidin D, hydroxyurea, idarubicin, ifosfamide, irinotecan, lomustine (CCNU), mechlorethamine, melphalan, 6-mercaptopurine, methotrexate, mithramycin, mitomycin C, mitoxantrone, nitroimidazole, paclitaxel, plicamycin, procarbizine, streptozotocin, tenoposide, 6-thioguanine, thioTEPA, topotecan, vinblastine, vincristine, vinorelbine, VP-16 and VM-26.

The conjugates of the invention used for enhancing the therapeutic effect of the protein of the invention also include non-classical therapeutic agents such as toxins. Such toxins include, for example, abrin, ricin A, bryodin, pseudomonas exotoxin, diphtheria toxin, saporin, a ribosome inactivating protein, or a porin protein, such as gonococcal PI porin protein. In another embodiment, the modified L49-sFv can be conjugated to a pro-drug converting enzyme. Examples of such enzymes are discussed in Section 5.1, supra.

Additional examples of therapeutic agents that can be conjugated to a modified L49-sFv of the invention include those described in International Patent Publication WO 02/088172, published on Nov. 7, 2002.

Techniques for conjugating such therapeutic moieties to proteins, and in particular to antibodies, are well known, see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc., 1985); Hellstrom et al., “Antibodies For Drug Delivery”, in Controlled Drug Delivery (2nd ed.), Robinson et al. (eds.), pp. 623-53 (Marcel Dekker, Inc., 1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., 1982, Immunol. Rev. 62, 119-58.

Alternatively, an antibody of the invention can be conjugated to a second antibody to form an antibody heteroconjugate as described by Segal in U.S. Pat. No. 4,676,980, which is incorporated herein by reference in its entirety.

5.3 Gene Therapy

In a specific embodiment, nucleic acids of the invention are administered to treat, inhibit or prevent a cancer which expresses p97 melanotransferrin, e.g., melanoma, and certain ovarian, breast, colon and lung cancers. Gene therapy refers to therapy performed by the administration to a subject of an expressed or expressible nucleic acid. In this embodiment of the invention, the nucleic acids produce their encoded protein that mediates a therapeutic effect, i.e., a modified L49-sFv of the present invention fused or conjugated to a therapeutic agent.

Any of the methods for gene therapy available in the art can be used according to the present invention. Exemplary methods are described below.

For general reviews of the methods of gene therapy, see, Goldspiel et al., 1993, Clinical Pharmacy 12, 488-505; Wu and Wu, 1991, Biotherapy 3, 87-95; Tolstoshev, 1993, Ann. Rev. Pharmacol. Toxicol. 32, 573-596; Mulligan, 1993, Science 260, 926-932; Morgan and Anderson, 1993, Ann. Rev. Biochem. 62, 191-217; May, 1993, TIBTECH 1, 1(5):155-215. Methods commonly known in the art of recombinant DNA technology which can be used are described in Ausubel et al. (eds.), Current Protocols in Molecular Biology, John Wiley & Sons, NY (1993); and Kriegler, Gene Transfer and Expression, A Laboratory Manual, Stockton Press, NY (1990).

In a preferred aspect, the therapeutic comprises nucleic acid sequences encoding a modified L49-sFv antibody, said nucleic acid sequences being part of expression vectors that express the modified L49-sFv antibody or fusion proteins thereof in a suitable host. In particular, such nucleic acid sequences have promoters operably linked to the antibody coding region, said promoter being inducible or constitutive, and, optionally, tissue-specific. In another particular embodiment, nucleic acid molecules are used in which the coding sequences and any other desired sequences are flanked by regions that promote homologous recombination at a desired site in the genome, thus providing for intrachromosomal expression of the encoding nucleic acids (Koller and Smithies, 1989, Proc. Natl. Acad. Sci. USA 86, 8932-8935; Zijlstra et al., 1989, Nature 342, 435-438. In specific embodiments, the expressed molecule is a modified L49 single chain antibody fused via a peptide bond to a pro-drug converting enzyme, e.g., β-lactamase.

Delivery of the nucleic acids into a patient may be either direct, in which case the patient is directly exposed to the nucleic acid or nucleic acid-carrying vectors, or indirect, in which case, cells are first transformed with the nucleic acids in vitro, then transplanted into the patient. These two approaches are known, respectively, as in vivo or ex vivo gene therapy.

In a specific embodiment, the nucleic acid sequences are directly administered in vivo, where it is expressed to produce the encoded product. This can be accomplished by any of numerous methods known in the art, for example by constructing them as part of an appropriate nucleic acid expression vector and administering the vector so that the nucleic acid sequences become intracellular. Gene therapy vectors can be administered by infection using defective or attenuated retrovirals or other viral vectors (see, e.g., U.S. Pat. No. 4,980,286); direct injection of naked DNA; use of microparticle bombardment (e.g., a gene gun; Biolistic, Dupont); coating with lipids or cell-surface receptors or transfecting agents; encapsulation in liposomes, microparticles, or microcapsules; administration in linkage to a peptide which is known to enter the nucleus; administration in linkage to a ligand subject to receptor-mediated endocytosis (see, e.g., Wu and Wu, 1987, J. Biol. Chem. 262, 4429-4432) (which can be used to target cell types specifically expressing the receptors); etc. In another embodiment, nucleic acid-ligand complexes can be formed in which the ligand comprises a fusogenic viral peptide to disrupt endosomes, allowing the nucleic acid to avoid lysosomal degradation. In yet another embodiment, the nucleic acid can be targeted in vivo for cell specific uptake and expression, by targeting a specific receptor (see, e.g., PCT Publications WO 92/06 180; WO 92/22635; WO92/20316; WO93/14188, and WO 93/20221). Alternatively, the nucleic acid can be introduced intracellularly and incorporated within host cell DNA for expression by homologous recombination (Koller and Smithies, 1989, Proc. Natl. Acad. Sci. USA 86, 8932-8935; Zijlstra et al., 1989, Nature 342, 435-438).

In a specific embodiment, viral vectors that contain nucleic acid sequences encoding an antibody of the invention are used. For example, a retroviral vector can be used (see Miller et al., 1993, Meth. Enzynmol. 217, 581-599). These retroviral vectors contain the components necessary for the correct packaging of the viral genome and integration into the host cell DNA. The nucleic acid sequences encoding the antibody to be used in gene therapy are cloned into one or more vectors, thereby facilitating delivery of the gene into a patient. More detail about retroviral vectors can be found in Boesen et al., 1994, Biotherapy 6, 29 1-302, which describes the use of a retroviral vector to deliver the mdr 1 gene to hematopoietic stem cells in order to make the stem cells more resistant to chemotherapy. Other references illustrating the use of retroviral vectors in gene therapy are: Clowes et al., 1994, J. Clin. Invest. 93, 644-651; Klein et al., 1994, Blood 83, 1467-1473; Salmons and Gunzberg, 1993, Human Gene Therapy 4, 129-141; and Grossman and Wilson, 1993, Curr. Opin. in Genetics and Devel. 3, 110-114.

Another approach to gene therapy involves transferring a gene to cells in tissue culture by such methods as electroporation, lipofection, calcium phosphate mediated transfection, or viral infection. Usually, the method of transfer includes the transfer of a selectable marker to the cells. The cells are then placed under selection to isolate those cells that have taken up and are expressing the transferred gene. Those cells are then delivered to a patient.

In this embodiment, the nucleic acid is introduced into a cell prior to administration in vivo of the resulting recombinant cell. Such introduction can be carried out by any method known in the art, including but not limited to transfection, electroporation, microinjection, infection with a viral or bacteriophage vector containing the nucleic acid sequences, cell fusion, chromosome-mediated gene transfer, microcell mediated gene transfer, spheroplast fusion, etc. Numerous techniques are known in the art for the introduction of foreign genes into cells (see, e.g., Loeffler and Behr, 1993, Meth. Enzymol. 217, 599-618; Cohen et al., 1993, Meth. Enzymol. 217, 618-644; Cline, 1985, Pharmac. Ther. 29, 69-92) and may be used in accordance with the present invention, provided that the necessary developmental and physiological functions of the recipient cells are not disrupted. The technique should provide for the stable transfer of the nucleic acid to the cell, so that the nucleic acid is expressible by the cell and preferably heritable and expressible by its cell progeny.

The resulting recombinant cells can be delivered to a patient by various methods known in the art. Recombinant blood cells (e.g., hematopoietic stem or progenitor cells) are preferably administered intravenously. The amount of cells envisioned for use depends on the desired effect, patient state, etc., and can be determined by one skilled in the art.

Cells into which a nucleic acid can be introduced for purposes of gene therapy encompass any desired, available cell type, and include but are not limited to fibroblasts; blood cells such as T lymphocytes, B lymphocytes, monocytes, macrophages, neutrophils, eosinophils, megakaryocytes, granulocytes; various stem or progenitor cells, in particular hematopoietic stem or progenitor cells, e.g., as obtained from bone marrow, umbilical cord blood, peripheral blood, fetal liver, etc.

In a preferred embodiment, the cell used for gene therapy is autologous to the patient.

In an embodiment in which recombinant cells are used in gene therapy, nucleic acid sequences encoding an antibody are introduced into the cells such that they are expressible by the cells or their progeny, and the recombinant cells are then administered in vivo for therapeutic effect. In a specific embodiment, stem or progenitor cells are used. Any stem and/or progenitor cells which can be isolated and maintained in vitro can potentially be used in accordance with this embodiment of the present invention (see e.g. PCT Publication WO 94/08598; Stemple and Anderson, 1992, Cell 71, 973-985; Rheinwald, 1980, Meth. Cell Bio. 21, A229; and Pittelkow and Scott, 1986, Mayo Clinic Proc. 61, 771).

In a specific embodiment, the nucleic acid to be introduced for purposes of gene therapy comprises an inducible promoter operably linked to the coding region, such that expression of the nucleic acid is controllable by controlling the presence or absence of the appropriate inducer of transcription.

The compounds or pharmaceutical compositions of the invention are preferably tested in vitro, and then in vivo for the desired therapeutic or prophylactic activity, prior to use in humans. For example, in vitro assays to demonstrate the therapeutic or prophylactic utility of an protein or pharmaceutical composition include determining the effect of the protein or pharmaceutical composition on a cancer cell line expressing p97 melanotransferrin or a tissue sample from a patient with a melanoma or a carcinoma expressing p97. The cytotoxic and/or cytostatic effect of the protein or composition on the cancer cell line and/or tissue sample can be determined utilizing techniques known to those of skill in the art. A preferred method, described in Section 6 infra, entails contacting a culture of the melanoma cell line grown at a density of approximately of about 5,000 cells/well in a 96 well plate with the modified protein or pharmaceutical composition, exposing the culture to the pro-drug C-Melphalan for 96 hours at 37° C., exposing the incubated cells with 10% alamarBlue™, a redox indicator, for 3 hours, and measuring the absorption at 570 nm. The protein or pharmaceutical composition has a cytostatic or cytotoxic effect on the cancer cell line and is useful for the treatment or prevention of cancer if the cells of the culture have reduced fluorescence at 570 nm compared to cells of the same cancer cell line cultured under the same conditions but not contacted with the protein or pharmaceutical composition. Alternatively, in vitro assays which can be used to determine whether administration of a specific protein or pharmaceutical composition is indicated, include in vitro cell culture assays in which a tissue sample from a cancer patient is grown in culture, and exposed to or otherwise a protein or pharmaceutical composition, and the effect of such compound upon the cancer tissue sample is observed.

5.4 Therapeutic/Prophylactic Administration and Composition

The invention provides methods of treatment and prophylaxis by administration to a subject of an effective amount of a modified L49-sFv of the invention fused or conjugated to a therapeutic agent which has a cytotoxic or cytostatic effect on cancer cells expressing p97 melanotransferrin (i.e., a protein of the invention), a nucleic acid encoding said L49-sFv protein (i.e., a nucleic acid of the invention), or a pharmaceutical composition comprising a protein or nucleic acid of the invention (hereinafter, a pharmaceutical of the invention). According to the present invention, treatment of cancer encompasses, in addition to its ordinary meaning, inhibition of progression of symptoms or amelioration of symptoms of a cancer, including a reduction in the size (volume) of a tumor and/or a reduction in the number of metastases.

In a preferred embodiment, the protein of the invention is a modified L49-sFv, wherein the modification is an amino acid substitution at position 40 of lysine for glutamine, at position 85 of phenylalanine for serine, and at position 95 of asparagine for tyrosine of SEQ ID NO:2 fused via a peptide bond to a pro-drug converting enzyme. In a preferred aspect, a pharmaceutical of the invention comprises a substantially purified protein or nucleic acid of the invention (e.g., substantially free from substances that limit its effect or produce undesired side-effects). The subject is preferably an animal, including but not limited to animals such as cows, pigs, horses, chickens, cats, dogs, etc., and is preferably a mammal, and most preferably human.

Formulations and methods of administration that can be employed are described above; additional appropriate formulations and routes of administration can be selected from among those described herein below.

Various delivery systems are known and can be used to administer a nucleic acid or protein of the invention, e.g., encapsulation in liposomes, microparticles, microcapsules, recombinant cells capable of expressing the compound, receptor-mediated endocytosis (see, e.g., Wu and Wu, 1987, J. Biol. Chem. 262, 4429-4432), construction of a nucleic acid as part of a retroviral or other vector, etc. Methods of introduction include but are not limited to intradermal, intramuscular, intraperitoneal, intravenous, subcutaneous, intranasal, epidural, and oral routes. Nucleic acids and proteins of the invention may be administered by any convenient route, for example by infusion or bolus injection, by absorption through epithelial or mucocutaneous linings (e.g., oral mucosa, rectal and intestinal mucosa, etc.) and may be administered together with other biologically active agents such as chemotherapeutic agents (see Section 5.8). Administration can be systemic or local.

In a specific embodiment, it may be desirable to administer the nucleic acid or protein of the invention by injection, by means of a catheter, by means of a suppository, or by means of an implant, said implant being of a porous, non-porous, or gelatinous material, including a membrane, such as a sialastic membrane, or a fiber. Preferably, when administering a protein, including an antibody, of the invention, care must be taken to use materials to which the protein does not absorb.

In another embodiment, the compound or composition can be delivered in a vesicle, in particular a liposome (see Langer, 1990, Science 249, 1527-1533; Treat et al., 1989, in Liposomes in the Therapy of Infectious Disease and Cancer, Lopez-Berestein and Fidler (eds.), Liss, New York, pp. 353-365; Lopez-Berestein, ibid., pp. 317-327; see generally, ibid.)

In yet another embodiment, the compound or composition can be delivered in a controlled release system. In one embodiment, a pump may be used (see Langer, supra; Sefton, 1989, CRC Crit. Ref. Biomed. Eng. 14, 201; Buchwald et al., 1980, Surgery 88, 507; Saudek et al., 1989, N. Engl. J. Med. 321, 574). In another embodiment, polymeric materials can be used (see Medical Applications of Controlled Release, 1974, Langer and Wise (eds.), CRC Pres., Boca Raton, Fla.; Controlled Drug Bioavailability, Drug Product Design and Performance, 1984, Smolen and Ball (eds.), Wiley, New York; Ranger and Peppas, 1983, Macromol. Sci. Rev. Macromol. Chem. 23, 61; see also Levy et al., 1985, Science 228, 190; During et al., 1989, Ann. Neurol. 25, 351; Howard et al., 1989, J. Neurosurg. 71, 105).

Other controlled release systems are discussed in the review by Langer, 1990, Science 249, 1527-1533.

In a specific embodiment where a nucleic acid of the invention is administered, the nucleic acid can be administered in vivo to promote expression of its encoded protein, by constructing it as part of an appropriate nucleic acid expression vector and administering it so that it becomes intracellular, e.g., by use of a retroviral vector (see U.S. Pat. No. 4,980,286), or by direct injection, or by use of microparticle bombardment (e.g., a gene gun; Biolistic, Dupont), or coating with lipids or cell-surface receptors or transfecting agents, or by administering it in linkage to a homeobox-like peptide which is known to enter the nucleus (see e.g., Joliot et al., 1991, Proc. Natl. Acad. Sci. USA 88, 1864-1868), etc. Alternatively, a nucleic acid can be introduced intracellularly and incorporated within host cell DNA for expression, by homologous recombination.

As alluded to above, the present invention also provides pharmaceutical compositions (pharmaceuticals of the invention). Such compositions comprise a therapeutically effective amount of a nucleic acid or protein of the invention, and a pharmaceutically acceptable carrier. In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a therapeutically effective amount of the nucleic acid or protein of the invention, preferably in purified form, together with a suitable amount of carrier so as to provide the form for proper administration to the patient. The formulation should suit the mode of administration.

In a preferred embodiment, the pharmaceutical of the invention is formulated in accordance with routine procedures as a pharmaceutical composition adapted for intravenous administration to human beings. Typically, compositions for intravenous administration are solutions in sterile isotonic aqueous buffer. Where necessary, the pharmaceutical of the invention may also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the pharmaceutical of the invention is to be administered by infusion, it can be dispensed with an infusion bottle containing sterile pharmaceutical grade water or saline. Where the pharmaceutical of the invention is administered by injection, an ampoule of sterile water for injection or saline can be provided so that the ingredients may be mixed prior to administration.

The amount of the nucleic acid or protein of the invention which will be effective in the treatment or prevention of cancer can be determined by standard clinical techniques. In addition, in vitro assays may optionally be employed to help identify optimal dosage ranges. See, Section 6.2.7 and 6.2.8, infra, for exemplary in vitro and in vivo assays. The precise dose to be employed in the formulation will also depend on the route of administration, and the stage of cancer, and should be decided according to the judgment of the practitioner and each patient's circumstances. Effective doses may be extrapolated from dose-response curves derived from in vitro or animal model test systems.

5.5 Kits

The invention also provides a pharmaceutical pack or kit comprising one or more containers filled with a nucleic acid or protein of the invention and optionally one or more pharmaceutical carriers. Optionally associated with such container(s) can be a notice in the form prescribed by a governmental agency regulating the manufacture, use or sale of pharmaceuticals or biological products, which notice reflects approval by the agency of manufacture, use or sale for human administration.

In one embodiment, a kit comprises a purified protein of the invention. In a preferred mode of the embodiment, the protein is an fusion protein. The protein may be conjugated or fused to a radionuclide or chemotherapeutic agent. The kit optionally further comprises a pharmaceutical carrier.

In another embodiment, a kit of the invention comprises a nucleic acid of the invention, or a host cell comprising a nucleic acid of the invention, operably linked to a promoter for recombinant expression.

5.6 Effective Dose

Toxicity and therapeutic efficacy of the proteins of the invention can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Proteins that exhibit large therapeutic indices are preferred. While proteins that exhibit toxic side effects may be used, care should be taken to design a delivery system that targets such proteins to the site of affected tissue in order to minimize potential damage to uninfected cells and, thereby, reduce side effects.

The data obtained from the cell culture assays and animal studies can be used in formulating a range of dosage for use in humans. The dosage of such proteins lies preferably within a range of circulating concentrations that include the ED₅₀ with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC₅₀ (i.e., the concentration of the test compound that achieves a half-maximal inhibition of symptoms) as determined in cell culture. Such information can be used to more accurately determine useful doses in humans. Levels in plasma may be measured, for example, by high performance liquid chromatography.

Generally, the dosage of a protein of the invention in a pharmaceutical of the invention administered to a cancer patient is typically 0.1 mg/kg to 100 mg/kg of the patient's body weight. Preferably, the dosage administered to a patient is between 0.1 mg/kg and 20 mg/kg of the patient's body weight, more preferably 1 mg/kg to 10 mg/kg of the patient's body weight.

5.7 Formulations

Pharmaceutical compositions for use in accordance with the present invention may be formulated in conventional manner using one or more physiologically acceptable carriers or excipients.

Thus, the proteins and their physiologically acceptable salts and solvates may be formulated for administration by inhalation or insufflation (either through the mouth or the nose) or oral, buccal, parenteral or rectal administration.

For oral administration, the pharmaceutical compositions may take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate) lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups or suspensions, or they may be presented as a dry product for constitution with water or other suitable vehicles before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate.

Preparations for oral administration may be suitably formulated to give controlled release of the active compound.

For buccal administration the compositions may take the form of tablets or lozenges formulated in conventional manner.

For administration by inhalation, the proteins for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, e.g., gelatin for use in an inhaler or insufflator may be formulated containing a powder mix of the compound and a suitable powder base such as lactose or starch.

The proteins may be formulated for parenteral administration by injection, e.g., by bolus injection or continuous infusion. Formulations for injection may be presented in unit dosage form, e.g, in ampoules or in multidose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

The proteins may also be formulated in rectal compositions such as suppositories or retention enemas, e.g., containing conventional suppository bases such as cocoa butter or other glycerides.

In addition to the formulations described previously, the proteins may also be formulated as a depot preparation. Such long acting formulations may be administered by implantation (for example subcutaneously or intramuscularly) or by intramuscular injection. Thus, for example, the proteins may be formulated with suitable polymeric or hydrophobic materials (for example as an emulsion in an acceptable oil) or ion exchange resins, or as sparingly soluble derivatives, for example, as a sparingly soluble salt.

The compositions may, if desired, be presented in a pack or dispenser device that may contain one or more unit dosage forms containing the active ingredient. The pack may for example comprise metal or plastic foil, such as a blister pack. The pack or dispenser device may be accompanied by instructions for administration preferably for administration to a human.

5.8 Combination Therapy for Treatment of Cancer

The nucleic acids and proteins of the invention can be administered together with treatment with irradiation or one or more chemotherapeutic agents.

For irradiation treatment, the irradiation can be gamma rays or X-rays. The treatment may comprise a single dose of irradiation or may comprise several doses of irradiation. The effective dose of irradiation can be calculated using methods known in the art taking into account the overall health of the patient and the type and location of the tumor. For a general overview of radiation therapy, see Hellman, Chapter 12: “Principles of Radiation Therapy Cancer”, in: Principles and Practice of Oncology, DeVita et al., eds., 2nd. Ed., J.B. Lippencott Company, Philadelphia.

Useful classes of chemotherapeutic agents include, but are not limited to, the following non-mutually exclusive classes of agents: alkylating agents, anthracyclines, antibiotics, antifolates, antimetabolites, antitubulin agents, auristatins, chemotherapy sensitizers, DNA minor groove binders, DNA replication inhibitors, duocarmycins, etoposides, fluorinated pyrimidines, lexitropsins, nitrosoureas, platinols, purine antimetabolites, puromycins, radiation sensitizers, steroids, taxanes, topoisomerase inhibitors, and vinca alkaloids. Individual chemotherapeutics encompassed by the invention include but are not limited to an androgen, anthramycin (AMC), asparaginase, 5-azacytidine, azathioprine, bleomycin, busulfan, buthionine sulfoximine, camptothecin, carboplatin, carmustine (BSNU), CC-1065, chlorambucil, cisplatin, colchicine, cyclophosphamide, cytarabine, cytidine arabinoside, cytochalasin B, dacarbazine, dactinomycin (formerly actinomycin), daunorubicin, decarbazine, docetaxel, doxorubicin, an estrogen, 5-fluordeoxyuridine, 5-fluorouracil, gramicidin D, hydroxyurea, idarubicin, ifosfamide, irinotecan, lomustine (CCNU), mechlorethamine, melphalan, 6-mercaptopurine, methotrexate, mithramycin, mitomycin C, mitoxantrone, nitroimidazole, paclitaxel, plicamycin, procarbizine, streptozotocin, tenoposide, 6-thioguanine, thioTEPA, topotecan, vinblastine, vincristine, vinorelbine, VP-16 and VM-26.

In a specific embodiment, a nucleic acid or protein of the invention is administered concurrently with radiation therapy or one or more chemotherapeutic agents. In another specific embodiment, chemotherapy or radiation therapy is administered prior or subsequent to administration of a nucleic acid or protein of the invention, by at least an hour and up to several months, for example at least an hour, five hours, 12 hours, a day, a week, a month, or three months, prior or subsequent to administration of a nucleic acid or protein of the invention.

In a specific embodiment in which a modified L49-sFv of the invention is conjugated or fused to a pro-drug converting enzyme, or in which a nucleic acid of the invention encodes a fusion protein comprising a modified L49-sFv and a pro-drug converting enzyme, the modified L49-sFv or nucleic acid is administered with a pro-drug. Administration of the pro-drug can be concurrent with administration of the nucleic acid or protein of the invention, or, more preferably, follows the administration of the nucleic acid or protein of the invention by at least an hour to up to one week, for example about five hours, 12 hours, or a day. Depending on the pro-drug converting enzyme administered, the pro-drug can be melphalan, a benzoic acid mustard, an aniline mustard, a phenol mustard, p-hydroxyaniline mustard-glucuronide, epirubicin-glucuronide, adriamycin-N phenoxyaceryl, N-(4′-hydroxyphenyl acetyl)-palytoxin doxorubicin, melphalan, nitrogen mustard-cephalosporin, β-phenylenediamine, vinblastine derivative-cephalosporin, cephalosporin mustard, cyanophenylmethyl-β-D-gluco-pyranosiduronic acid, 5-(adaridin-1-yl-)2,4-dinitrobenzamide, or methotrexate-alanine.

The invention is further described in the following examples which are in no way intended to limit the scope of the invention.

### 6. EXAMPLE

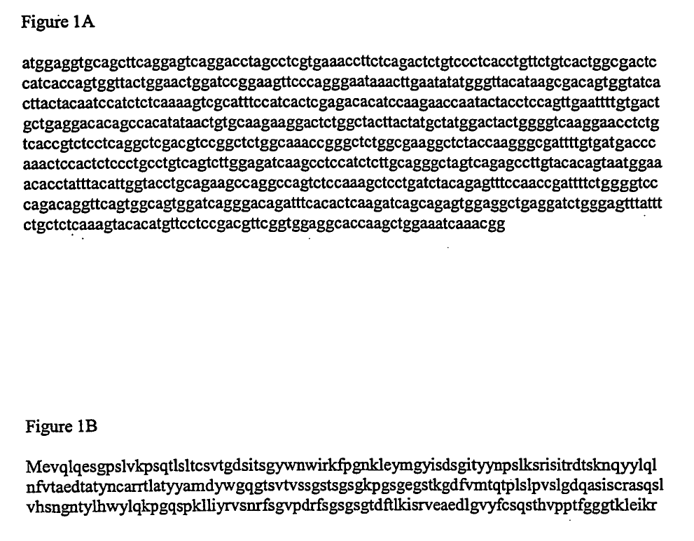

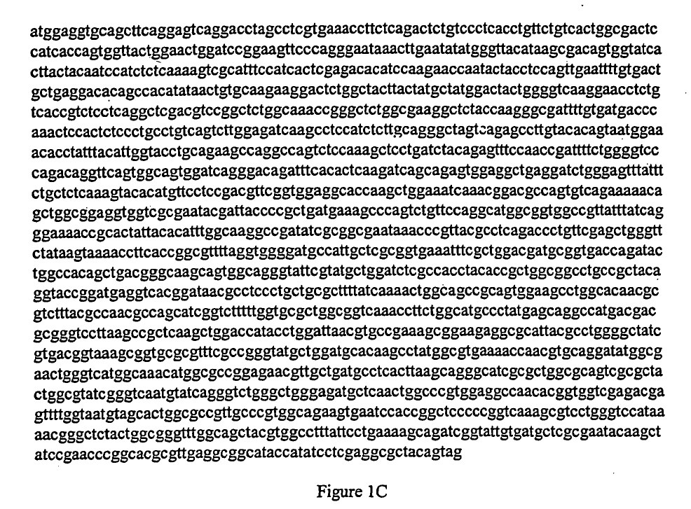

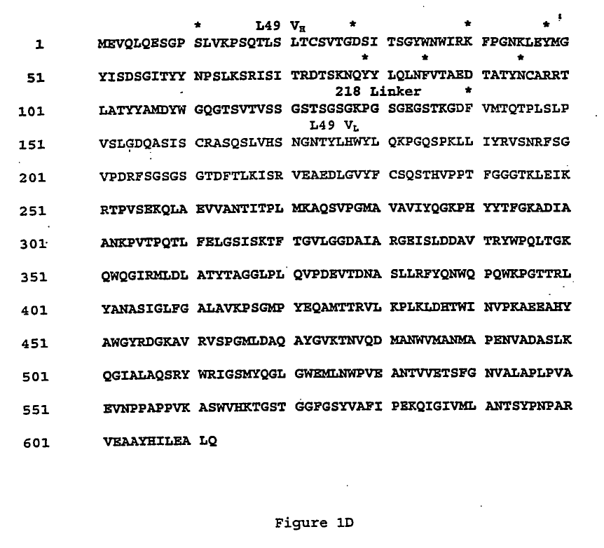

The following describes a sequence alignment approach to re-engineer L49-sFv-bL. FIGS. 1A-1B depicts the nucleotide sequence (SEQ ID NO:1) (FIG. 1A) and its encoded amino acid sequence (SEQ ID NO:2) (FIG. 1B) of the parental L49-sFv molecule. FIG. 1C depicts the nucleotide sequence (SEQ ID NO:3) of the parental L49-sFv-bL molecule and FIG. 1D depicts the amino acid sequence of the parental L49-sFv-bL (SEQ ID NO:4), including the wild type heavy and light chain variable regions, the linker separating the chains and the β-lactamase sequence. The asterisks (*) indicate the residues that are mutated to provide the novel modified L49-sFv molecules of the present invention. This method rapidly identified three residues in the V_{H} framework that, when mutated to consensus residues, improved both the yield and stability of refolded L49-sFv-bL. Binding affinity and efficacy were unaffected by the introduction of these stabilizing mutations. Further, the effects of orientation on yield were also investigated and the results show that positioning the β-lactamase protein at the amino terminus (bL-L49-sFv) increased protein yields several-fold compared to L49-sFv-bL where the β-lactamase fusion is positioned at the carboxyl terminus.

6.1 Materials and Methods

6.1.1 Mutagenesis of L49-scFv

Mutagenesis was performed by PCR overlap extension using pfu turbo polymerase (Stratagene). The template used was the previously described L49-scFv-bL construct (Siemers et al., 1997, Bioconjug. Chem. 8, 510-519) cloned into pET27b (Novagen). Mutagenic primers used were: sense primer 5′ACCAAGGGCGATGTTGTGATGACCCAA (SEQ ID NO:5) and anti-sense primer 5!TTGGGTCATCACAACATCGCCCTTGGT (SEQ ID NO:6) for F L2 V; sense primer 5′GAGTCAGGACCTGGCCTCGTGAAACCT (SEQ ID NO:7) and anti-sense primer 5′AGGTTTCACGAGGCCAGGTCCTGACTC (SEQ ID NO:8) for S H10 G; sense primer 5′TCTGTCACTGGCTACTCCATCACCAGT (SEQ ID NO:9) and anti-sense primer 5′ACTGGTGATGGAGTAGCCAGTGACAGA (SEQ ID NO:10) for D H27 Y; sense primer 5′AACTGGATCCGGCAGTTCCCAGGGAAT (SEQ ID NO:11) and anti-sense primer 5′ATTCCCTGGGAACTGCCGGATCCAGTT (SEQ ID NO: 12) for K H39 Q; sense primer 5′GGGAATAAACTTGAATGGATGGGTTACATAAGC (SEQ ID NO:13) and anti-sense primer 5′GCTTATGTAACCCATCCATTCAAGTTTATTCCC (SEQ ID NO:14) for Y H47 W; sense primer 5′TCCAAGAACCAAGCCTACCTCCAGTTG (SEQ ID NO:15) and antisense primer 5′CAACTGGAGGTAGGCTTGGTTCTTGGA (SEQ ID NO:16) for Y H78 A; sense primer 5′CTCCAGTTGAATTCTGTGACTGCTGAG (SEQ ID NO: 17) and anti-sense primer 5′CTCAGCAGTCACAGAATTCAACTGGAG (SEQ ID NO:18) for F H82B S; and sense primer 5′ACAGCCACATATTACTGTGCAAGAAGG (SEQ ID NO:19) and anti-sense primer 5′CCTTCTTGCACAGTAATATGTGGCTGT (SEQ ID NO:20) for N H91 Y (characters in bold are mutated codon). Flanking primers 5′GGATCGAGATCTCGATCCCGCGAAATT (sense) (SEQ ID NO:21) containing a BgIII site (underlined) and 5′GCCTGGCTTCTGCAGGTACCAATGTAAATA (antisense) (SEQ ID NO:22) containing a Pst I site (underlined) were used to amplify mutated PCR products. Products were digested with Bgl II and Pst I (New England Biolabs) and cloned into the Bgl II/Pst I fragment of the original pET27b L49-scFv-bL construct replacing the parental sequence. Mutants were confirmed by sequencing. The reverse orientation construct FP999 was made by amplifyig the β-lactamase cDNA from pET27b L49-scFv-bL incorporating a 5′ NcoI site and a 3′ HindIII site within a synthetic 6 amino acid linker (MHGTKL (SEQ ID NO:23)). The cDNA coding for L49-sFv was amplified from the expression construct incorporating a 5′ HindIII site and a 3′ NheI site. PCR products were digested with NcoI and HindIII and HindIII and NheI respectively and cloned into pET27b digested with NcoI and NleI.

6.1.2 Protein Expression and Purification

For small scale expression BL21([DE3) (Novagen) were transformed with parental and mutant constructs and single colonies was used to inoculate 100 ml Terrific Broth II (TB) (QBIOGENE). Cells were induced at an OD of 1.0 with 1 mM IPTG (Sigma). Cells were harvested following overnight incubation and inclusion bodies were purified from the cell extracts using B-Per (Novagen) according to the manufacturers instructions. Inclusion body pellets were resuspended in 8 M urea, 2 mM DTT, 50 mM Tris.Cl pH 8.0 at a concentration of 50 mg/ml. Inclusion bodies were refolded overnight in a 50-fold dilution of 2 M urea, 0.3 M L-arginine, 50 mM Tris.Cl pH 8.0, 1 mM reduced glutathione, 0.1 mM oxidized glutathione. Protein was then dialyzed into PBS and fusion protein concentration was determined by measuring β-lactamase activity using colorimetric substrate nitrocefin.

For western analysis proteins were run on a 10% Tris/Glycine gel (Novagen) and transferred to nitrocellulose. Blots were probed with a 1:5000 dilution of affinity purified rabbit polyclonal anti-serum to E. cloacae β-lactamase (Siemers et al., 1997, Bioconjug. Chem. 8, 510-519) and then blocked with 1% BSA in PBS. Secondary HRP-conjugated anti-rabbit IgG was applied at 1:2000 dilution and following wash steps blots were developed using colorimetric substrate 3,3′-daiaminobenzidine tetrahydochloride (DAB) (Sigma).

For large scale expression 50 ml of TB were inoculated with transformed BL21 (DE3). After an OD₆₀₀ of 0.6-0.8 was reached the entire 50 mL culture was used to inoculate 500 mL of TB and grown at 37° C. with shaking in two liter flasks. At an OD₆₀₀ of 1 cultures were induced with 1 mM IPTG and grown overnight. Cells were harvested at 5000 rpm for 10 minutes and 10 g of wet cell pellets were suspended in 500 mL of TE buffer (50 mM Tris, 1 mM EDTA, pH 8) containing 1% v/v Triton X-100 (Fisher Biotech). The suspension was passed 3 times through a gaulin homogenizer at 6000-8000 psi and inclusion bodies were collected via centrifugation. The resulting pellet was washed 2× with TE+1% TX-100 and an additional 2× with TE alone. Washed inclusion bodies were immediately solublized using a stator/rotor homogenizer at a concentration of 50 mg wet IB weight/mL in 10 M Urea, 50 mM TRIS pH 8 containing 2 mM DTT and incubated for 1 hour at room temperature with gentle rotation. After reduction solublized inclusion bodies were filtered and concentration of protein determined by absorbance at 280 nm using an extinction coefficient of 1.9. Solublized inclusion bodies were immediately diluted 1:50 v/v into cold (2-8° C.) refolding buffer containing 2 M Urea, 0.3 M L-Arginine, 50 mM TRIS, pH 8, 1 mM reduced glutathione, 0.1 mM oxidized glutathione, with stirring. Refolding was allowed to continue for 72-96 hours at 2-8° C.

500 mL of refolded fusion protein was diluted 1:3 with PBS containing additional 0.5 M NaCl then vacuum filtered through 0.21 μm bottle top filter. Diluted/filtered refold was applied to a 17 mL aminophenylboronate column (either Millipore PROSEP-PB, or Prometic Biosciences) at 1.5 mL/min previously equilibrated with 5 column volumes (CV) of equilibration buffer. The column was washed with 5 CV of equilibration buffer and bound protein was eluted with 50 mM Diethanolamine, 0.5 M NaCl pH 11.2. The eluate was immediately neutralized with 30:1 v/v 1M Tris.Cl pH 7.5. Neutralized PBA eluate was dialyzed against 10 mM sodium phosphate pH 7. The dialysate was filtered and bound to Macro-Prep HS (BioRad, Hercules, Calif.) previously equilibrated in 10 mM sodium phosphate, pH 7. The bound protein was washed with 5 CV of 10 mM sodium phosphate, pH 7 and eluted with 10 mM sodium phosphate pH 7, 250 mM NaCl.

HPLC-SEC analysis was performed on an HP Agilent HPLC system. Purified L49-sFv-bL was assayed on a 7.8×300 mm TSK G3000swxl (TosoHaas, Pa.) employing an isocratic gradient of PBS as the mobile phase at 1 ml/min. Recording was performed at 280 nm.

6.1.3 P97 Binding Assays

Soluble p97 (amino acids 20-710 of human p97 precursor) was cloned into pSecTag2 (Invitrogen), incorporating an Ig Kappa leader sequence and a 6× his tag, and transfected into CHO cells. Stable transformants were selected and secreted protein was purified from conditioned medium on a metal chelate affinity column. Assays were performed by coating polystyrene 96-well plates with 2 μg/ml soluble p97 in PBS overnight. The plates were blocked by adding 1% bovine serum albumin in PBS for 1 hour at room temperature. Plates were emptied and fresh blocking reagent containing serial dilutions of L49-sFv-bL samples was added. Following 1 hour at room temperature the plates were washed and developed with 0.1 ml of colorimetric β-lactamase substrate nitrocefin solution at 0.1 mM in PBS containing 1% DMSO. Absorbance measurements were read in an ELISA plate reader using a 490 nm filter with 610 m as the reference wavelength.

Binding affinity was measured at 25° C. on a Biacore 2000 (Biacore analysis was performed by the Protein Interaction Facility at the University of Utah). The buffer used in the study was PBS, 0.005% P20, 0.2 mg/mi BSA. Antigen was immobilized on one flow cell surface of a CM5 sensor chip and the others were left blank to serve as reference surfaces. For each scFv, triplicates of 0, 0.5, 2.8, 8.3, 25, 75 and 225 nM were injected across the immobilized antigen. The association and dissociation phases were monitored for 1 and 20 minutes, respectively. The antigen surface was regenerated with four 18-second pulses of 2.5 mM NaOH between injections.

6.1.4 Stability Assays

Stability assays were performed by incubating samples at 25 μg/ml in mouse plasma at 37° C. At each time point, 0, 24, 48 and 72 hours, an aliquot was removed and immediately frozen at −80° C. At the end of the experiment all aliquots were thawed and analyzed for binding to p97 using the ELISA-based assay.

6.1.5 In Vitro Cytotoxicity Assay

H3677 cells were plated into 96 well microtiter plates (5×10³ cells/well in 100 μl of RPMI 1640 media (Invitrogen) with 10% fetal bovine serum) and allowed to adhere overnight. The cells were treated with FP95, FP990 or FP999 fusion (see Table 2) proteins at 10 nM. After 1 hour at 4° C. the plates were washed three times with medium and then concentrations of pro-drug C-Mel from 0.005 nM-100 nM were added. Melphalan was also added at the same concentrations to cells treated with medium alone. After 1 hour at 37° C. cells were washed three times with medium and incubated for 96 hours at 37° C. The cells were then incubated with 10% alamarBlue™ for 3 hours at 37° C. and excitation was measured in an ELISA plate reader at 570 nm.

6.1.6 In Vivo Therapy Experiments

In vivo studies were performed in female BALB/c nude mice, which were 5-6 weeks of age at the start of each study. H3677 human melanoma tumors were established as subcutaneous xenografts in donor mice and serially passaged into recipients for study. When tumors averaged approximately 100 mm³ the mice were randomized into groups of five and received either no treatment, C-Mel alone, or L49-sFv-bL (derived from either FP990 or FP999) at 1 mg/kg followed 24 h later by C-Mel at the indicated doses. Tumor-volume, in mm³, was defined as (length×width²)/2. Both L49-sFv-bL and C-Mel were administered intravenously via the tail vein.

6.2 Results

6.2.1 Identification of Unusual Residues in the Framework Region of L49

To limit the effect of mutagenesis on binding affinity, CDR regions of the L49 mAb were not considered for alteration. Using the method described by Chowdhury et al., 1998, J. Mol. Biol. 281, 917-928 seven significant differences were observed between L49 V_{H} framework and consensus sequences at positions H10 (S), H27 (D), H39 (K), H47 (Y), H78 (Y), H82B (F) and H91 (N) and one significant difference was found between the L49 V_{L} framework and its consensus at position L2 (F) (Kabat numbering). The frequency of occurrence of these residues at these positions in the entire Kabat database (Johnson and Wu, 2001, Nucleic Acids Res. 29, 205-206), which currently contains greater than 14000 V_{H} and 6000 Kappa V_{L} sequences, was determined as described by Chowdhury et al., 1998, J. Mol. Biol. 281, 917-928. All these residues were found to occur in less than 3% of variable domain sequences. Further investigation of the V_{H} positions revealed that H39, H47 and H91 were involved in forming the interface between the V_{H} and V_{L} domains (Chothia et al., 1985, J. Mol. Biol. 186, 651-663). We speculated that the presence of unusual amino acids in these positions disrupted the V_{H}/V_{L} interface resulting in an unstable molecule. The hydrophobic phenylalanine at position H82B is incompatible with its predicted exposed surface accessibility (Chowdhury et al., 1998, J. Mol. Biol. 281, 917-928), which may induce instability. Table 1 summarizes the positions in the heavy chain and light chain that were identified as having an unusual amino acid.

TABLE 1

| | | Position | L49-sFv- | | | Most |
|---|---|---|---|---|---|---|
| | Position | in L49- | bL Amino | % | Surface | com- |
| | (Kabat) | sFv-bL | acid | Occurrence | accessibility | mon |
| V_{H} | H10 | 11 | S | 2-3% | | G |
| | H27 | 28 | D | 1-2% | b | Y |
| | H39 | 40 | K | 2-3% | L | Q |
| | H47 | 48 | Y | 1-2% | L | W |
| | H78 | 79 | Y | 1-2% | b | A |
| | H82B | 85 | F | <1% | e | S |
| | H91 | 95 | N | <1% | L | Y |
| V_{L} | L2 | 140 | F | <1% | b | I |

% occurrence = frequency in Kabat database as reported by Chowdhury et al.
Surface accessibility = as reported by Chowdhury et al.(b = buried, e = exposed and L = contact with V_{L} interface)
Most common = as reported by Chowdhury et al.

6.2.2 Analysis of L49 scFv Framework Mutants by Measurement of p97 Binding Activity

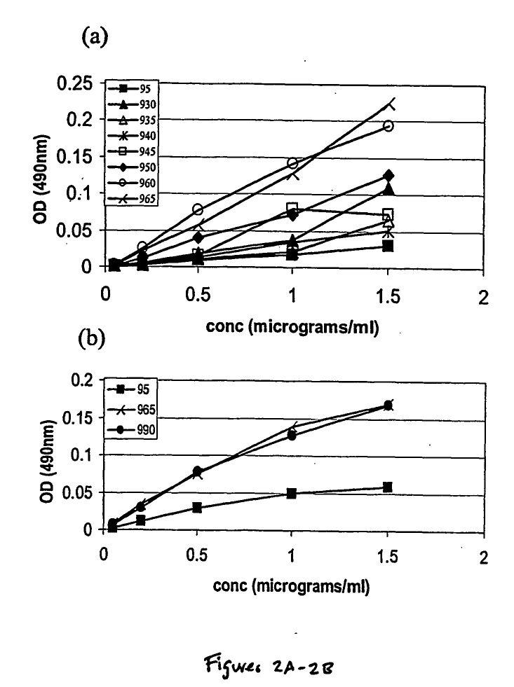

The unusual residues in the L49 scFv framework were mutated to the most frequently occurring residue for that position in the Kabat database (Chowdhury et al., 1998, J. Mol. Biol. 281, 917-928). The following individual mutations were introduced into the parental L49-sFv-bL (FP95): S H10 G, (FP920), D H27 Y (FP925), K H39 Q, (FP930), Y H47 W, (FP935), Y H78 A, (FP940), F H82B S (FP945), N H91 Y (FP950), F L2 I (FP955) (Table 2). To efficiently assay for improved refolding characteristics, the antigen binding activities of the refolded L49-sFv-bL mutants were compared to the refolded parental molecule (FP95). The parental and mutant fusion proteins were expressed in E. coli as inclusion bodies and denatured and refolded as described in Section 6.1.2, supra. The crude refolds were normalized based on β-lactamase activity. Binding to p97 was then determined using a solid-phase binding assay as described in Section 6.1.3, supra. V_{H} mutants at positions H39, H82B and H91 showed significant increases in antigen binding compared to the parental L49-sFv-bL molecule, indicating improved refolding and stability, with mutant N H91 Y having the most pronounced effect (FIG. 2A). The V_{L} single mutant, F L2 I, and VH single mutants S H10 G and D H27 Y showed no improvement in antigen binding compared to the parental molecule (data not shown). The three most effective heavy chain mutations were combined to create the following double and triple mutants: F H82B S, N H91 Y (FP960), K H39 Q, N H91 Y (FP965) and K H39 Q, F H82B S, N H91 Y (FP990) (Table 2). Both the double mutants and triple mutant displayed similar improvements in antigen binding activity over the single mutants (FIGS. 2A-2B).

TABLE 2

| L49-sFv | | | wt amino | mutant |
|---|---|---|---|---|
| fusion protein | Kabat position | Orientation | acid | amino acid |
| FP95 | wt | L49-sFv-bL | wt | wt |
| FP920 | H10 | L49-sFv-bL | S | G |
| FP925 | H27 | L49-sFv-bL | D | Y |
| FP930 | H39 | L49-sFv-bL | K | Q |
| FP935 | H47 | L49-sFv-bL | Y | W |
| FP940 | H78 | L49-sFv-bL | Y | A |
| FP945 | H82B | L49-sFv-bL | F | S |
| FP950 | H91 | L49-sFv-bL | N | Y |
| FP960 | H82B/H91 | L49-sFv-bL | F/N | S/Y |
| FP965 | H39/H91 | L49-sFv-bL | K/N | Q/Y |
| FP990 | H39/H82B/H91 | L49-sFv-bL | K/F/N | Q/S/Y |
| FP999 | H39/H82B/H91 | bl-L49-sFv | K/F/N | Q/S/Y |

6.2.3 Western Analysis of L49-sFv-bL Mutants

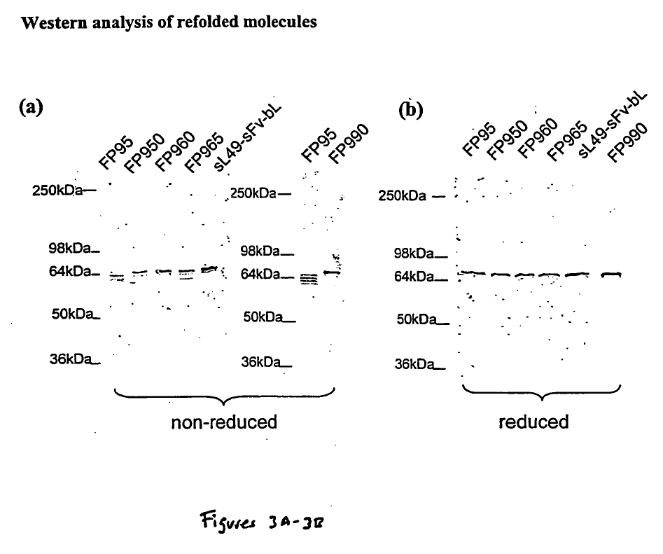

Western blot analysis of non-reduced crude refolds with polyclonal anti-β-lactamase antibody showed that the parental L49-sFv-bL molecule (FP95) exists as several different species, indicating the presence of misfolded protein (FIG. 3A). Under reducing conditions only one band is observed (FIG. 3B). The solubly expressed parental molecule, sL49-sFv-bL, which is active both in vitro and in vivo (Siemers et al., 1997, Bioconjug. Chem. 8, 510-519) resolves to only one band under non-reducing conditions (FIG. 3A). Mutant FP950 and double mutants FP960 and FP965 also resolve to one main band (FIG. 3A), which suggests that the majority of protein refolds into the active conformation. The triple mutant, FP990, resolves completely to one band (FIG. 3A) suggesting that the introduction of all three V_{H} mutations results in the most improved refolding characteristics.

6.2.4 Expression, Purification and Analysis of Parental and Mutated L49-sFv-bL Fusion Proteins

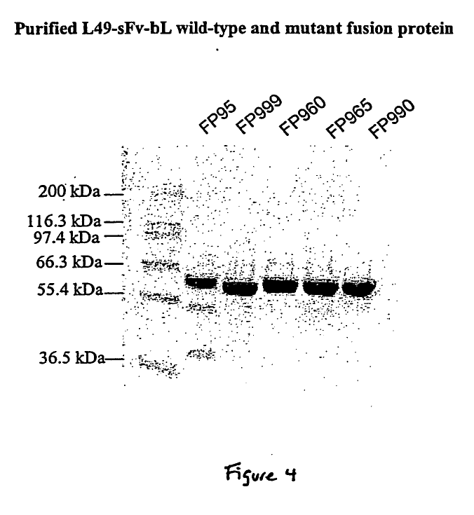

Double mutants FP960 and FP965 and triple mutant FP990 all show improved refolding activity in crude lysates compared to the parental molecule, and therefore, all three mutant molecules were selected for further analysis. Proteins were expressed at the 1L scale in shake flasks as described in Materials and Methods, supra. Inclusion bodies (IBs) were obtained at a typical yield of 25% (±10%) wet IB mass/wet cell mass. Renatured L49-sFv-bL was purified by aminophenylboronate chromatography, dialyzed into 10 mM Na₂PO₄ and loaded onto Macro-prep HS resin. Fusion proteins were eluted in a stepwise fashion employing 250 mM NaCl. Yields of the mutated constructs showed a significant improvement compared to the parental molecule with increased yields for FP960, FP965 and FP990 of 7.5-fold, 4.8-fold and 8.2-fold respectively (Table 3). Mutants FP965 and FP990 were greater than 95% monomeric as shown by SEC-HPLC. However, FP960 contained only 55% monomer after cation exchange purification, possibly indicating a key role for the lysine residue at position H39 in refolding. The parental molecule, FP95, was present as 2 bands following aminophenylboronate chromatography. After ion-exchange chromatography only the upper of these 2 bands was eluted. A size exclusion chromatography (SEC) binding assay was developed in which a 2:1 ratio of purified p97 antigen to purified L49-sFv-bL was mixed and analyzed by HPLC-SEC (data not shown). This assay showed that the eluted upper band bound antigen completely demonstrating that it contains the active refolded protein. All mutants resolved as one homogenous band during the purification process (FIG. 4) and bound antigen completely in the SEC assay.

The effect of orientation on protein yield was investigated by reversing the order of the molecule so that β-actamase was present at the amino terminus. As β-actamase is a highly stable, well-expressed molecule positioning it at the amino terminus might drive expression of the fusion protein resulting in higher yields. The three V_{H} mutations were introduced into this reverse orientation molecule to produce mutant FP999. This strategy was successful, resulting in almost 20-fold increased expression compared to the parental molecule. This was due to an increase in protein expression as shown by the increase in recovery of inclusion body protein (Table 3). Purified FP999 was also greater than 95% monomeric following cation exchange purification and bound completely to antigen.

TABLE 3
Typical expression and purification yields

| | | Fusion Protein | | | | |
|---|---|---|---|---|---|---|
| Step | Recovery | FP95 | FP960 | FP965 | FP990 | FP999 |
| Fermentation | g/L of culture | 9.7 | 10.6 | 11.5 | 10.3 | 8.6 |
| Inclusion body prep. | IB, g | 3.0 | 2.6 | 2.9 | 3.1 | 3.5 |
| | % recovery IB/g cell | 31 | 24 | 25 | 30 | 41 |
| Solubilization | Total mg | 208 | 178.6 | 148 | 188 | 764.5 |
| Final yield | mg/L culture | 4.8 | 36 | 23.1 | 39.2 | 95 |

6.2.5 Binding Properties of Parental and Mutant L49-sFv-bL Fusion Proteins

The binding affinities of double mutants FP960 and FP965 and triple mutants FP990 and FP999 were compared to the parental molecule by surface plasmon resonance. Recombinant p97 was immobilized on a BIACore CM5 sensor chip and varying concentrations of each scFv fusion protein were injected across the chip in triplicate. The K_{D} values for all four mutant molecules were similar to the parental molecule, FP95, showing that introduction of the three V_{H} framework mutations does not adversely affect the structure of the CDR loops (Table 4). These values compared favorably with previously published data, which reported a K_{D} for the parental L49-sFv-bL molecule, expressed in the E. coli periplasm, of 1 nM using similar techniques (Siemers et al., 1997, Bioconjug Chem. 8, 510-519).

TABLE 4
Binding Affinity of mutant L49-sFv-bL parental
and mutant molecules as determined by surface plasmon resonance

| Sample | ka (M⁻¹ s⁻¹) | kd (s⁻¹) | KD (nM) |
|---|---|---|---|
| FP95 | 3.038(2) × 10⁵ | 7.004(5) × 10⁻⁴ | 2.306(1) |
| FP960 | 3.640(4) × 10⁵ | 6.383(7) × 10⁻⁴ | 1.754(1) |
| FP965 | 4.000(6) × 10⁵ | 7.65(1) × 10⁻⁴ | 1.91(1) |
| FP990 | 4.476(1) × 10⁵ | 7.819(3) × 10⁻⁴ | 1.747(1) |
| FP999 | 3.373(3) × 10⁵ | 5.755(7) × 10⁻⁴ | 1.706(1) |

Values shown are the average of triplicate readings. The data was fit to a 1:1 interaction model to obtain the shown kinetic and affinity parameter. Numbers shown in parentheses are the error in the last digit.

6.2.6 Stability of Parental and Mutant L49-sFv-bL in Mouse Plasma

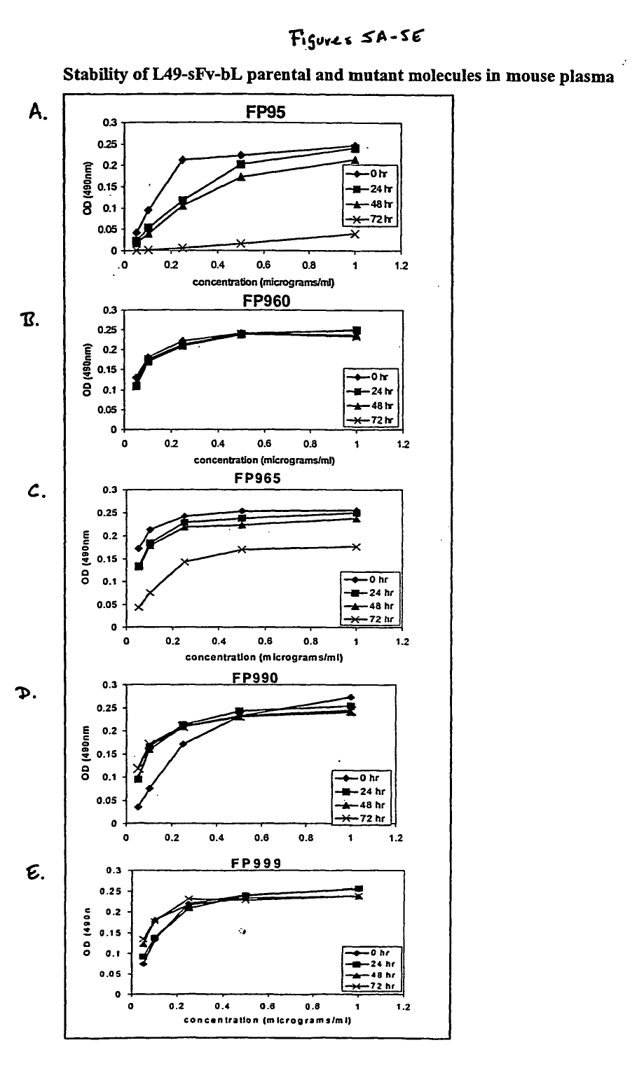

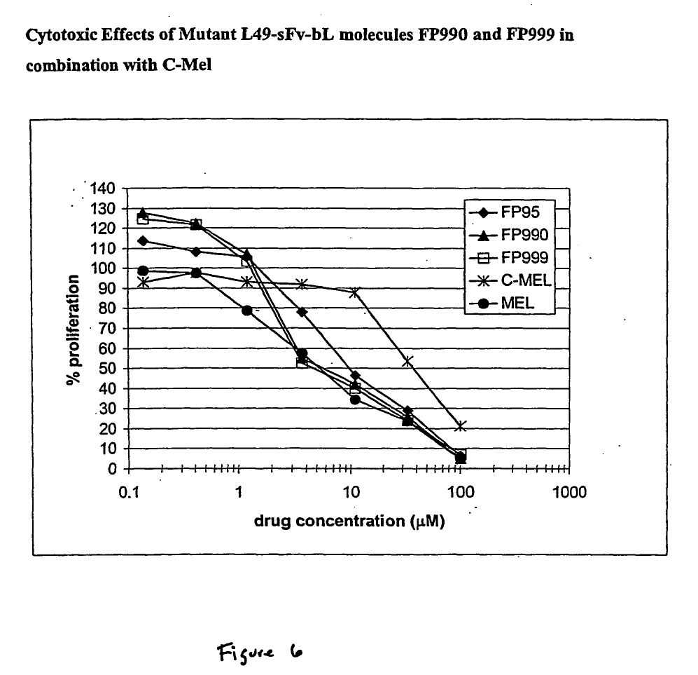

An effective agent for ADEPT needs to remain stable at the site of the tumor for many hours to several days to allow a high tumor to blood ratio to be established before application of the prodrug (Senter and Springer, 2001, Adv. Drug Deliv. Rev. 53, 247-264). The parental L49-sFv-bL molecule has been shown to clear rapidly from the systemic circulation with a terminal half-life t_{1/2}β=2.5 hours. A high tumor to blood ratio of 141-150:1 was measured 24-48 hours after the fusion protein was administered (Siemers et al., 1997, Bioconjug Chem. 8, 510-519). To ensure that the mutated L49-sFv-bL proteins remain stable in the tumor environment for an extended time period each mutant and the parental molecule was incubated in mouse plasma at 37° C. at a concentration of 25 μg/ml. Aliquots were removed at 0, 24, 48 and 72 hours and tested for binding to antigen using the solid phase p97 binding assay. Mutants FP960, FP990 and FP999 were highly stable retaining 100% antigen binding activity throughout the 72 hour period. The parental molecule, FP95, lost 84% antigen binding activity between 48 and 72 hours and mutant FP965 lost 31% activity during the same time period (FIG. 5).

6.2.7 Cytotoxicity of L49-sFv-bL Parental and Mutant Molecules

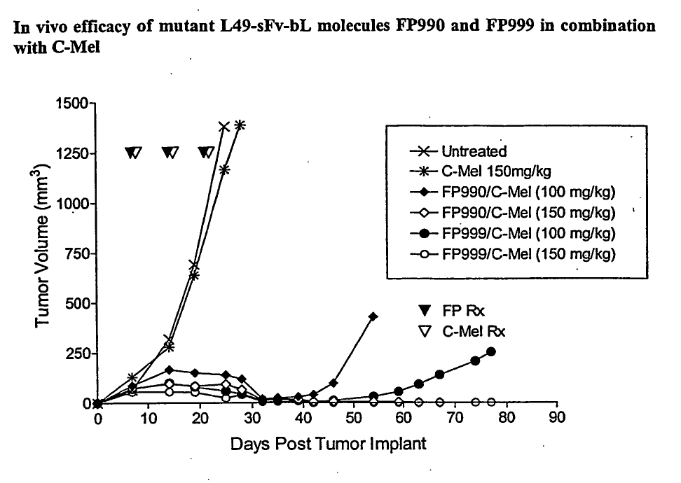

Results from the stability studies and the properties of the mutant molecules during purification indicated that the triple mutant molecules in each orientation, FP990 and FP999, were the most suitable candidates for further study. Therefore, these two mutants and the parental molecule were tested in combination with the prodrug C-Mel, a cephalosporin-containing derivative of melphalan (Kerr et al., 1998, Bioconjug Chem. 9, 255-259), for their effectiveness in killing cultured human melanoma H3677 cells, which express the p97 antigen. The cells were treated with 10 nM L49-sFv-bL, unbound material was washed off and serial dilutions of C-Mel were added. C-Mel and the active drug, melphalan, were also applied to cells treated with media alone. Cell viability was measured using the redox indicator alamarBlue™. The IC50 for C-Mel was 40 μM while the combination of C-Mel and mutants FP990 or FP999 were equally effective in prodrug conversion resulting in an IC₅₀ of 5 μM, equivalent to the activity of melphalan (FIG. 6).

6.2.8 In Vivo Efficacy of L49-sFv-bL Mutants

The relative efficacy of the two triple mutants, in combination with C-Mel, was compared in H3677 human melanoma xenografts in athymic nude mice. This L49 antigen-positive model was previously evaluated with the parental L49-sFv-bL fusion construct and was shown to be sensitive when combined with C-Mel (Kerr et al., 1998, Bioconjug Chem. 9, 255-259) and CCM, a prodrug of phenylenediamine mustard (Siemers et al., 1997, Bioconjug. Chem. 8, 510-519). The study was initiated with implantation, on day 0, of solid tumor obtained from donor mice and therapy was initiated on day 7, when the tumors averaged approximately 100 mm³. Groups of mice (5/group) received either no therapy (untreated controls) or one of five therapeutic regimens: C-Mel alone at 150 mg/kg, or mutant FP990 or FP999 at 1 mg/kg followed 24 h later by C-Mel at either 100 or 150 mg/kg. Both components were administered every seven days for a total of three injections (q7dx3) with the fusion protein administered on days 7, 14, and 21, and C-Mel administered on days 8, 15 and 22. Treatment with either mutant L49-sFv-bL molecule and C-Mel at 150 mg/kg led to complete tumor regression in all of the animals (FIG. 7.). Both fusion proteins also gave significant tumor regression in combination with C-Mel at 100 mg/kg At the end of the study there were two complete regressions in the FP999 group but none in the FP990 group. With the limited number of animals used in this study the difference between these two groups is not significant. In contrast to the animals that received both L49-sFv-bL and C-Mel, C-Mel alone had no anti-tumor activity, indicating that the anti-tumor effect was specific to the combination of fusion protein and prodrug. These results show that the FP990 and FP999 constructs are highly effective when combined with prodrug C-Mel.

6.3 Discussion

The effective anti-tumor activity of the combination of L49-scFv-bL fusion protein with cephalosporin-containing nitrogen-mustard prodrugs C-Mel or CCM in mouse melanoma models has been previously reported (Siemers et al., 1997, Bioconjug. Chem. 8, 510-519; Kerr et al., 1998, Bioconjug Chem. 9, 255-259). However, development of this treatment for clinical use has been hindered by difficulties in producing sufficient quantities of L49-sFv-bL. The parental L49-sFv-bL molecule, expressed in denatured inclusion bodies, refolds inefficiently into native active protein able to bind to antigen, although the β-lactamase moiety retains activity. Therefore, it appears that the scFv portion of the molecule is responsible for the observed refolding problems. Our goal was to use a protein engineering approach to improve the refolding properties of L49-sFv-bL and increase protein yields to allow for development of this molecule into a clinical candidate.

6.3.1 Identification of Unusual Residues in L49-scFv

Analysis of refolded mutant molecules revealed three effective mutations in the heavy chain. Two of the residues identified, H39 (K) and H91 (N), are in positions involved in formation of the V_{H}/V_{L} interface (Chothia et al., 1985, J. Mol. Biol. 186, 651-663). Position H39 is a glutamine in 97% of all mouse V_{H} domains and a lysine is present in this position in the remaining 3% of sequences (Johnson and Wu, 2001, Nucleic Acids Res. 29, 205-206). The glutamine side chain at position H39 forms two hydrogen bonds across the V_{H}/V_{L} interface with a highly conserved glutamine at position L38. In L49 the presence of a lysine at position H39 of the V_{H} domain allows only one hydrogen bond to form with the glutamine at L38 instead of the two hydrogen bonds made between two glutamine side chains, thus reducing the stability of the interaction. Mutation of the conserved glutamine at H39 to an alanine within Fab 57P resulted in reduced antigen binding activity despite its location at least 9 Å away from the base of the CDR loops (Chatellier et al., 1996, J. Mol. Biol. 264, 1-6). Another study, performed on a scFv, showed that increasing hydrophobic interactions at the V_{H}/V_{L} interface by mutating either H39 or L38 or both to methionine actually increases the refolding efficiency and stability while reducing antigen binding (Tan et al., 1998, Biophys. J. 75, 1473-1482). Both these studies highlight the importance of the interface interaction between H39 and L38 and suggest a role for these residues in both antigen binding and folding stability.

The residue at position H91 is a tyrosine or occasionally a tryptophan in the majority of all V_{H} domains. An asparagine occurs in this position, as is the case in L49, in only 0.025% of heavy chains. H91 contacts both L43 and L44 across the V_{H}/V_{L} interface (Chothia et al., 1985, J. Mol. Biol. 186, 651-663) and mutation of tyrosine at this position to alanine has been shown to eliminate antigen binding and result in reduced expression, suggesting reduced stability (Chatellier et al., 1996, J. Mol. Biol. 264, 1-6). The presence of a non-aromatic asparagine residue at position H91 in L49 may disrupt interactions with the V_{L} domain at positions L43 and L44, destabilizing the V_{H}/V_{L} interface and causing misfolding of the scFv. In the whole L49 antibody this destabilization is probably countered by the presence of the constant domains.

The third unusual residue in the V_{H} domain occurs at position H82B close to the region of the variable/constant domain interface in the whole antibody (Nieba et al., 1997, Protein Eng 10, 435-444). The L49 residue at this position is an aromatic henylalanine, while in the majority of V_{H} sequences a small, relatively hydrophilic serine is resent. Since position H82B is generally exposed at the scFv surface (Chowdhury et al., 1998, J. Mol. Biol. 281, 917-928), a phenylalanine at this position creates a hydrophobic patch, which may induce protein aggregation. Due to the close proximity of position H82B to the variable/constant interface this phenylalanine may be buried in the L49 antibody becoming exposed and problematic only in the scFv. Mutagenesis of hydrophobic residues formerly present at the variable/constant interface to hydrophilic residues was shown to increase the functional expression of an anti-fluorescein scFv fragment 25-fold (Nieba et al., 1997, Protein Eng 10, 435444) highlighting the negative effect of hydrophobic surface residues on protein stability.

6.3.2 Mutagenesis of Unusual Residues in L49-sFv-bL and Characterization of Mutants

Three single V_{H} mutants, FP930, FP945 and FP950 improved the refolding and antigen binding characteristics of the L49-sFv-bL molecule. Combinations of these three effective V_{H} mutants improved refolding activity further although the triple mutant FP990 showed no improvement over double mutants FP960 and FP965 when crude refolds were analyzed. Multiple disulfide-bonded species of parental L49-sFv-bL, FP95, are observed under non-reducing conditions indicating that the majority of protein is misfolded. Similar multiple bands have also been observed for another scFv fusion protein, G28-5 scFv-PE40 (Francisco et al., 1995, Cancer Res. 55, 3099-3104). In this case only one of the bands could be purified by affinity chromatography over immobilized antigen suggesting that the remaining protein was an incorrect disulfide species. The introduction of the V_{H} mutations into L49-sFv-bL encouraged the formation of one disulfide species, presumably active refolded protein, with the triple V_{H} mutant, FP990, having the most pronounced effect suggesting that all three mutations are required for optimal refolding. We found that the yields of purified double mutants, FP960 and FP965 and triple mutant, FP990, were 4.8-8.2-fold greater than for the parental, FP95, and 100% of the purified protein bound to antigen. Purified FP960, however, was found to contain high molecular weight aggregate, which may be due to the wild type lysine residue remaining at position H39. Mutation of the conserved glutamine at position H39 of Fab 57P to alanine also resulted in formation of high molecular weight aggregates indicating that a glutamine is preferred at this position (Chatellier et al., 1996, J. Mol. Biol. 264, 1-6). However, FP960 did remain stable during extended stability studies at 37° C. in mouse plasma while the other double mutant FP965 did not, losing activity between 48 and 72 hours. When yields of purified protein, western analysis and stability studies are compared it is clear that all three V_{H} mutations are preferred for optimal refolding and stability.

Interestingly, the parental molecule, FP9S, loses 84% of its activity between 48 and 72 hours in mouse plasma while FP960, FP990 and FP999 remain stable so introduction of the V_{H} mutations not only increases yield through more efficient refolding but also increases stability and extends the effective time period for application of prodrug. Surprisingly, introduction of the V_{H} mutations did not decrease antigen binding affinity, as has been reported following scFv engineering (Chowdhury et al., 1998, J. Mol. Biol. 281, 917-928). The three V_{H} mutations functioned effectively to improve the stability of the framework structure while maintaining the binding integrity of the CDR loops. We speculate that introduction of the stabilizing mutations at the V_{H}/V_{L} interface prevents disassociation of the V_{H} and V_{L} domains during refolding steps, which would expose hydrophobic residues and promote aggregation. Likewise replacement of phenylalanine at position H82B with serine removes another hydrophobic patch from the scFv surface. As the parental molecule misfolds into several different disulfide-bonded species the V_{H} mutations must also contribute to stabilization of the V_{H} intradomain disulfide bond.

6.3.3 Effect of Reversing the Orientation of the L49-sFv-bL Fusion Protein

β-lactamases are highly stable, well-expressed bacterial enzymes that have been fused to other proteins at both their N and C terminus without affecting enzyme activity (Francisco et al., 1992, Proc. Natl. Acad. Sci. U.S.A. 89, 2713-2717; Lattemann et al., 2000, J. Bacteriol. 182, 3726-3733). Highly expressed proteins, such as glutathione-S-transferase, are commonly fused to the N terminus of less well-expressed proteins to drive expression and increase yield. Reversing the orientation of L49-sFv-bL and fusing β-lactamase to the N terminus of L49-scFv also had this effect with yields of FP999, containing the three V_{H} mutations, 2.5-fold greater than its counterpart, FP990. Antigen binding affinity (Table 4) and enzyme activity (data not shown) were unaffected by the change in orientation and stability was similar to FP990.

6.3.4 Efficacy of the Re-Engineered L49-sFv-bL Molecule

As discussed, the mutations introduced into the L49-sFv-bL molecule do not adversely affect antigen-binding affinity and, as would be expected, the re-engineered molecule remains as efficacious as the original L49-sFv-bL molecule. This is also true for the reverse orientation molecule FP999. In vivo therapy studies in nude mice with subcutaneous H3677 tumors using either FP990 or FP999 in combination with C-Mel were comparable to a previous study performed with the original L49-sFv-bL molecule using an almost identical protocol (Kerr et al., 1998, Bioconjug Chem. 9, 255-259). One therapeutic advantage of the engineered L49-sFv-bL molecule is its improved stability as assessed at 37° C. in mouse plasma. This may extend the presence of active L49-sFv-bL at the tumor and allow multiple doses of C-Mel to be applied following a single injection of L49-sFv-bL. In single dose experiments C-Mel was most effective at 150 mg/kg concentrations, which is only half its MTD. Administering multiple lower doses of C-Mel following a single dose of L49-sFv-bL may be equally effective and, as C-Mel is likely to be rapidly cleared from circulation, improve the therapeutic window.

In conclusion, mutating only three unusual amino acids in the scFv portion of L49-sFv-bL has considerably increased the yield of active refolded protein and improved the stability of the molecule without effecting therapeutic efficacy. These improvements have allowed clinical development of the L49-sFv-bL/C-Mel combination to be considered.

The present invention is not to be limited in scope by the specific embodiments described herein. Indeed, various modifications of the invention in addition to those described herein will become apparent to those skilled in the art from the foregoing description and accompanying figures. Such modifications are intended to fall within the scope of the appended claims.

Various patent and non-patent publications are cited herein, the disclosures of which are incorporated herein by reference in their entireties.

We claim:

1. A modified L49 sFv, wherein the modification is an amino acid substitution at position 85 (Kabat position H82B) of phenylalanine for serine, threonine or alanine of SEQ ID NO:2.

2. A modified L49 sFv, wherein the modification is an amino acid substitution at position 95 (Kabat position H91) of asparagine for tyrosine or phenylalanine of SEQ ID NO:2.

3. A modified L49 sFv, wherein the modification is an amino acid substitution at position 85 (Kabat position H82B) of phenylalanine for serine, threonine or alanine, and at position 95 (Kabat position H91) of asparagine for tyrosine or phenylalanine of SEQ ID NO:2.

4. A modified L49 sFv, wherein the modification is an amino acid substitution at position 40 (Kabat position H39) of lysine for glutamine, and at position 95 (Kabat position H91) of asparagine for tyrosine or phenylalanine of SEQ ID NO:2.

5. A modified L49 sFv, wherein the modification is an amino acid substitution at position 40 (Kabat position H39) of lysine for glutamine, and at position 85 (Kabat position H82B) of phenylalanine for serine, threonine or alanine of SEQ ID NO:2.

6. A modified L49 sFv, wherein the modification is an amino acid substitution at position 40 (Kabat position H39) of lysine for glutamine, at position 85 (Kabat position H82B) of phenylalanine for serine, threonine or alanine, and at position 95 (Kabat position H91) of asparagine for tyrosine or phenylalanine of SEQ ID NO:2.

7. The modified L49 sFv according to claim 1, 2, 3, 4, 5 or 6, which is fused via a peptide bond to a therapeutic agent.

8. The modified L49 sFv according to claim 7, in which the therapeutic agent is a cytotoxic molecule or a pro-drug converting enzyme.

9. The modified L49 sFv according to claim 8, in which the cytotoxic molecule or pro-drug converting enzyme is fused via a peptide bond to the N-terminus of said modified L49 sFv.

10. The modified L49 sFv according to claim 8, in which the cytotoxic molecule or pro-drug converting enzyme is fused via a peptide bond to the C-terminus of said modified L49 sFv.

11. The modified L49 sFv according to claim 8, in which the cytotoxic molecule is selected from the group consisting of abrin, ricin A, bryodin, pseudomonas exotoxin, diphtheria toxin, saporin, and a porin protein.

12. The modified L49 sFv according to claim 8, in which the pro-drug converting enzyme is selected from the group consisting of Herpes simplex thymidine 15 kinase, bacterial cytosine deaminase, cytochrome p450 NADPH oxidoreductase, alkaline phosphatase, α-galactosidase, β-galactosidase, aminopeptidase, aryl sulfatase, glucose oxidase, caspase, carboxylesterase, xanthine oxidase, elastase, nitroreductase, carboxypeptidase G2, beta-glucuronidase, penicillin-V-amidase, penicillin-G-amidase, β-lactamase, β-glucosidase, and carboxypeptidase A.

13. The modified L49-sFv according to claim 8, in which the pro-drug converting enzyme is beta-lactamase.

14. The modified L49 sFv according to any one of claims 1-13, in which the modified L49-sFv is purified.

15. The modified L49 sFv according to claim 1, 2, 3, 4, 5 or 6, which is conjugated to a therapeutic agent.

16. The modified L49 sFv according to claim 15, in which the therapeutic agent is a cytotoxic molecule or a pro-drug converting enzyme.

17. A pharmaceutical composition comprising (a) a purified, modified L49 according to any of claims 7-13; and (b) a pharmaceutically acceptable carrier.

18. A nucleic acid comprising a nucleotide sequence encoding the modified L49 sFv according to any of claims 1-6.

19. A nucleic acid comprising a nucleotide sequence encoding the modified L49 sFv according to any of claims 7-13.

20. A nucleic acid comprising:
(a) the nucleotide sequence of SEQ ID NO:1 in which nucleotide residues 253-255 are changed to AGT, AGC, TCT, TCC, TCA, TCG, ACC, ACA, ACG, ACT, GCC, GCA, GCG or GCT;
(b) the nucleotide sequence of SEQ ID NO:1 in which nucleotide residues 283-285 are changed to TAC, TAT, TTC or TTT;
(c) the nucleotide sequence of SEQ ID NO:1 in which nucleotide residues 253-255 are changed to AGT, AGC, TCT, TCC, TCA, TCG, ACC, ACA, ACG, ACT, GCC, GCA, GCG or GCT, and nucleotide residues 283-285 are changed to TAC, TAT, TTC or UTT;
(d) the nucleotide sequence of SEQ ID NO:1 in which nucleotide residues 118-120 are changed to CAG or CAA, and nucleotide residues 283-285 are changed to TAC, TAT, TTC or TUT;
(e) the nucleotide sequence of SEQ ID NO:1 in which nucleotide residues 118-120 are changed to CAG or CAA, and nucleotide residues 253-255 are changed to AGT, AGC, TCT, TCC, TCA, TCG, ACC, ACA, ACG, ACT, GCC, GCA, GCG or GCT; and
(f) the nucleotide sequence of SEQ ID NO: 1 in which nucleotide residues 118-120 are changed to CAG or CAA, nucleotide residues 253-255 are changed to AGT, AGC, TCT, TCC, TCA, TCG, ACC, ACA, ACG, ACT, GCC, GCA, GCG or GCT, and nucleotide residues 283-285 are changed to TAC, TAT, TTC or TTT.

21. The nucleic acid according to claim 18, 19 or 20, which is isolated.

22. A recombinant vector comprising the nucleic acid according to claim 18, 19 or 20 operably linked to a promoter.

23. A host cell comprising the recombinant vector according to claim 22.

24. A method for producing a modified L49 sFv comprising culturing the host cell of claim 23 such that the nucleic acid is expressed by the cell to produce its encoded modified L49 sFv molecule; and isolating the expressed molecule.

25. A pharmaceutical composition comprising (a) a purified nucleic acid according to claim 19; and (b) a pharmaceutically acceptable carrier.

26. A method for treating or preventing cancer, wherein the cancer expresses p97 melanotransferrin comprising administering to a subject in need of such treatment or prevention, an effective amount of a pharmaceutical composition according to claim 17 or 25.

27. A method for treating or preventing cancer, wherein the cancer expresses p97 melanotransferrin comprising administering to a subject in need of such treatment or prevention, (a) an effective amount of a pharmaceutical composition comprising a purified modified L49-sFv according to any of claims 1-6 fused or conjugated to a pro-drug converting enzyme; and (b) an effective amount of a pro-drug that is converted to its active form by said pro-drug converting enzyme.

28. The method according to claim 26 or 27, in which the cancer is a melanoma.

29. The method according to claim 26 or 27, in which the cancer is breast cancer, lung cancer, ovarian cancer, renal cancer or colon cancer.

30. A method for treating or preventing cancer, wherein the cancer expresses p97 melanotransferrin comprising administering to a subject in need of such treatment or prevention, (a) an effective amount of a pharmaceutical composition comprising a purified modified L49-sFv according to any of claims 1-6 fused to a therapeutic agent, wherein said therapeutic agent is selected from the group consisting of an androgen, anthramycin (AMC), asparaginase, auristatin, 5-azacytidine, azathioprine, bleomycin, busulfan, buthionine sulfoximine, camptothecin, carboplatin, carmustine (BSNU), CC-1065, chlorambucil, cisplatin, colchicine, cyclophosphamide, cytarabine, cytidine arabinoside, cytochalasin B, dacarbazine, dactinomycin, daunorubicin, decarbazine, docetaxel, doxorubicin, an estrogen, 5-fluordeoxyuridine, 5-fluorouracil, gramicidin D, hydroxyurea, idarubicin, ifosfamide, irinotecan, lomustine (CCNU), mechlorethamine, melphalan, 6-mercaptopurine, methotrexate, mithramycin, mitomycin C, mitoxantrone, nitroimidazole, paclitaxel, plicamycin, procarbizine, streptozotocin, tenoposide, 6-thioguanine, thioTEPA, topotecan, vinblastine, vincristine, vinorelbine, VP-16 and VM-26.

31. A molecule comprising the modified L49-sFv according to any of claims 1-13.

32. A molecule comprising the nucleic acid according to any one of claims 18-20.

33. The molecule according to claim 31 or 32 which is purified.
